(19) United States
(12) Patent Application Publication
(10) Pub. No.: US 2018/0221299 A1
(43) Pub. Date: Aug. 9, 2018

(54) TRANSDERMAL DELIVERY SYSTEM

(71) Applicant: EURO-CELTIQUE S.A., Cambridgeshire (LU)

(72) Inventors: Steve WHITELOCK, Cambridge (GB); Deborah Phyllis HARDING, Cambridge, Cambridgeshire (GB); Helen JOHNSON, Cambridge (GB); Kevin SMITH, Cambridge, (GB); Gill MUNDIN, Cambridge (GB)

(73) Assignee: EURO-CELTIQUE S.A., Cambridgeshire (LU)

(21) Appl. No.: 15/748,548

(22) PCT Filed: Jul. 28, 2016
(86) PCT No.: PCT/GB2016/052307

§ 371 Date: Jan. 29, 2018

(30) Foreign Application Priority Data
Jul. 30, 2015 (GB) 1513441.4

(51) Int. Cl. A61K 9/70; A61K 9/00; A61K 31/485; A61P 25/04

(52) U.S. Cl. CPC A61K 9/7061; A61P 25/04; A61K 31/485; A61K 9/0014

(57) ABSTRACT

The present invention provides a transdermal delivery system comprising (R)-dihydroetorphine, or a salt, hydrate or derivative thereof, wherein said system has a rapid onset of (R)-dihydroetorphine plasma concentration characterised by the mean in vivo plasma concentration of (R)-dihydroetorphine achieving at least 50% of its Cₘₐₓ in less than 20 hours, preferably in less than 18 hours and more preferably in less than 12 hours, after application of the system to the skin of a human subject, e.g. when based on the mean plasma concentration versus time curve.

### INTRODUCTION

The present invention relates to a transdermal delivery system and in particular to a transdermal patch comprising (R)-dihydroetorphine, or a salt, hydrate or derivative thereof, which, when applied to the skin of a human subject provides a rapid delivery of (R)-dihydroetorphine into the plasma and whereupon removal from the skin achieves a rapid decrease in the concentration of (R)-dihydroetorphine in the plasma. The invention is also concerned with the use of a transdermal system in medicine and in particular in a method of providing pain relief or analgesia.

### BACKGROUND

Pain, which can be acute or chronic, is the most common symptom for which patients seek medical advice and treatment. Acute pain is usually self-limited. Chronic pain persists for 3 months or longer and can lead to significant changes in a patient's personality, lifestyle, functional ability and overall quality of life (K. M. Foley, Pain, in Cecil Textbook of Medicine 100-107 (J. C. Bennett and F. Plum eds., 20th ed. 1996)). Pain can also be classified into different acute, subacute and chronic types including nociceptive, inflammatory, neuropathic or mixed pain.

Pain relief occurs in different clinical settings and is critical in the management and treatment of many diseases wherein pain is experienced as a symptom and/or as a side effect. Opioid analgesics form the cornerstone of contemporary treatment of moderate to severe, acute and chronic, pain. The opioid analgesics that are most commonly used to treat pain include morphine, hydromorphone, methadone, levorphanol, fentanyl, oxycodone, and oxymorphone.

In many circumstances it is necessary to provide pain relief for a prolonged or sustained period of time. Sustained pain relief is particularly desirable in patients suffering from moderate to severe chronic pain, e.g. cancer patients. Oral formulations can provide a therapeutic analgesic effect for up to 12, or in a few cases, up to 24 hours but such formulations still require the drug to be readministered at least once or twice a day.

Another approach to sustained delivery of drugs, including analgesics, is transdermal delivery systems such as transdermal patches. Transdermal patches typically comprise a therapeutically active ingredient (e.g. an opioid), an adhesive, optionally a matrix, a backing layer and a release liner. The release liner is removed prior to application of the patch to the skin to expose the adhesive. The adhesive enables the patch to adhere to the skin thereby allowing for passage of the active ingredient from the patch through the skin and into the blood stream.

Transdermal patches have numerous advantages over other routes of administration. These include:
the treatment is comfortable, non-invasive, pain free and convenient
the treatment is well tolerated with high compliance rates
the treatment can potentially be self-administered once patients have been educated on patch use and disposal
the treatment provides a more constant blood concentration of active ingredient than other routes which avoids frequent dosing
the treatment is ongoing regardless of the time of day
the treatment enables a high level of control over the blood concentration of the drug
the drug bypasses the gastrointestinal tract and the liver where it can be destroyed and instead is delivered to the blood stream
the effects of the drug can be terminated by removal of the patch

Many patent applications and literature articles describe patches comprising opioids and in particular buprenorphine and fentanyl. For example, US2007/0298091 describes patches comprising buprenorphine and WO2009/052204 and US2006/0039960 and WO2005/105009 each disclose patches comprising fentanyl.

Two transdermal patches comprising an opioid are commercially available. The BuTrans® or Norspan® patch, for example, comprises 5 mg, 10 mg, or 20 mg of buprenorphine (a partial opioid agonist) and delivers 5 μg/h, 10 μg/h or 20 μg/h over a period of 7 days. It is indicated for the treatment of non-malignant pain of moderate intensity when an opioid is necessary for obtaining adequate analgesia. The Durogesic® Dtrans® patch comprises 2.1, 4.2, 8.4, 12.6 and 16.8 mg of fentanyl and is indicated for the management of chronic pain including chronic pain due to cancer.

The development of commercially viable transdermal patches that provide controlled and sustained release of a drug is not straightforward. To achieve the benefits of transdermal delivery, a transdermal patch that is stable and is able to achieve a sufficient flux of drug through the skin is necessary. It is critical that the drug, and the other constituents, of the transdermal patch does not undergo degradation or change during storage or use. For example, it is important that the drug remain dissolved within the patch throughout its lifetime in order to be deliverable through the skin. Otherwise the flux of drug through the skin will be inconsistent.

The stability of a drug in a transdermal patch is highly dependent on the nature of the drug and the nature of the patch. The structure of the drug, and its chemical and physical properties, has a significant influence on stability, flux and its interaction with any polymers it is formulated with. It is not therefore possible to substitute one opioid for another opioid in a patch and obtain a commensurate performance. Each drug requires the development of a suitable transdermal patch.

It is also important that the flux of drug through the skin and into the blood stream can be maintained for a prolonged period of time and ideally at least 3 days for a number of the above-described advantages (e.g. high compliance, infrequent dosing, ongoing treatment) of transdermal delivery to be fully realised. To achieve this it is common to include additional ingredients such as permeation enhancers and permeation sustaining agents into transdermal patches to improve control over the permeation of drug. The inclusion of additional ingredients into transdermal patches, however, makes provision of a stable patch yet more complex since the constituents are prone to interacting with the drug. To overcome this problem it is common to provide the drug in specific drug-reservoir layers which are separated from other ingredients to minimise the contact of the drug with them.

A wide range of opioid analgesics are known. Opioid agonists include, for example, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydrocodone, hydromorphone, hydromorphodone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, pantopon, papavereturn, paregoric, pentazocine, phenadoxone, phendimetrazine, phendimetrazone, phenomorphan, phenazocine, phenoperidine, piminodine, piritramide, propheptazine, promedol, properidine, propoxyphene, propylhexedrine, sufentanil, tilidine, tramadol and pharmaceutically acceptable salts thereof. To date, only buprenorphine and fentanyl have been formulated into commercially available transdermal patches.

Another known opioid analgesic is (R)-dihydroetorphine (R-DHE) (CAS No. 14357-76-7). Its chemical name is 7,8-dihydro-7a-[1-(R)-hydroxy-1-methylbutyl]-6,14-endo-ethanotetrahydro-oripavine. Its stereochemical configuration is with 5R, 6R, 7R, 9R, 13S, 14S, 19R and it is shown below.

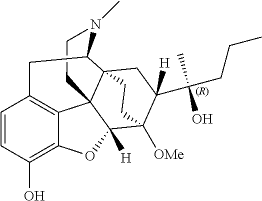

The properties of (R)-dihydroetorphine have been investigated to a far lesser extent than the properties of other opioid analgesics. Clinically it has only been used in humans in China in injectable, and more recently, sublingual form.

There are also relatively few literature reports on the use of (R)-dihydroetorphine. US2005/002997 discloses a transdermal dosage form comprising both a drug and an antagonist to minimise abuse of the dosage form. A long list of possible drugs is disclosed including dihydroetorphine, but, as in the prior art documents mentioned above, the focus of US2005/002997 is on fentanyl. The transdermal dosage form disclosed in US2005/002997 specifically requires the drug to be separated from the adverse agent. Thus typically there exists a drug-containing layer and an adverse agent layer, separated by a barrier which prevents diffusion of the drug and the adverse agent in the absence of solvent. Thus in normal transdermal use, only the drug is transdermally delivered. The drug-containing layer is also required to comprise at least one channel which connects the skin contacting surface with the barrier. The channel enables solvent (e.g. saliva or solvent) to access the adverse agent layer in the event an abuser attempts to extract drug from the transdermal patch. Notably there is no transdermal delivery data in US2005/002997 for a dihydroetorphine-containing patch.

JP-A 10-231248 to TTS Gijutsu Kenkyusho KKrefers to a prototype transdermal device comprising dihydroetorphine and a styrene-isoprene-styrene block copolymer. More specifically JP-A 10-231248 refers to a tape for percutaneous absorption which comprises dihydroetorphine and a styrene-isoprene-styrene block copolymer. The purpose of the preparations in JP-A 10-231248 is said to be to provide a sustained therapeutic effect. This is preferably achieved by including a percutaneous absorption enhancer and a percutaneous absorption-sustaining agent in the preparation. The effect of the percutaneous absorption enhancer is to accelerate percutaneous absorption and the effect of the percutaneous absorption-sustaining agent is to sustain absorption.

In the examples of JP-A 10-231248 some preparations are prepared and the rate of dihydroetorphine release is measured. There is, however, no disclosure of a patch which provides prolonged delivery of dihydroetorphine for a clinically useful period of time, e.g. at least 3 days. JP-A 10-231248 does not therefore disclose a clinically useful transdermal patch

We have found that when prototype transdermal patches comprising a drug-containing layer of (R)-dihydroetorphine and styrene-isoprene-styrene block copolymer, as illustrated in JP-A 10-231248, were prepared and tested, the (R)-dihydroetorphine was found to be highly unstable. Under forced conditions, designed to replicate long-term storage, it was found that (R)-dihydroetorphine, in the presence of styrene-isoprene-styrene block copolymer, had a strong tendency to crystallise out in the drug-containing layer. This is highly undesirable since it was found that the (R)-dihydroetorphine will not redissolve once crystallised. When in crytallised form, however, the (R)-dihydroetorphine is unavailable for transdermal delivery through the skin. Consequently the permeation and flux of (R)-dihydroetorphine is decreased.

Two literature articles disclose basic dihydroetorphine containing patches. Chen et al. in Acta Pharmaceutica Sinica 1996 31 (10), 770-774 disclose a patch comprising a dihydroetorphine layer as well as a separate adhesive layer. The adhesive layer primarily comprises polyvinyl alcohol, polyvinyl pyrrolidone, lactose and azone. In the study described patches having a size of 1 cm² and comprising 5 μg dihydroetorphine were applied to Wistar rats. The blood concentration of dihydroetorphine achieved was monitored over time. The conclusion reached in the study is that dihydroetorphine may be delivered stably for a period of about 30 hours.

Ohmori et al. in J. Pharm. Pharmacol. 2000 52, 1437-1449 describe a study on the transdermal delivery from a patch comprising dihydroetorphine and a styrene-isoprene-styrene lock copolymer in rats. In the study, patches having a size of 0.28 cm² or 0.50 cm² and comprising 20 μg or 35 μg of dihydroetorphine respectively were applied to the abdominal region or dorsal region of rats. The patches were removed after 8 hours (abdomen) or 24 hours (dorsal). The resulting dihydroetorphine plasma concentration curve was measured over a short period of 32 hours. The corresponding analgesic effect was measured by the tail immersion test. The conclusion reached by the study is that dihydroetorphine is permeable enough through the skin of hairless rats to achieve an analgesic effect. It is also noted, however, that the plasma concentration of dihydetorphine is relatively variable and it is speculated that this is due to variation of drug input rate through the skin which, in turn, was influenced by the cutaneous perfusion rate and expansion and contraction of skin in contact with the patches. Neither Chen nor Ohmori disclose a transdermal patch comprising dihydroetorphine which is clinically useful for treatment of humans.

### SUMMARY OF INVENTION

Viewed from a first aspect, the present invention provides a transdermal delivery system comprising (R)-dihydroetorphine, or a salt, hydrate or derivative thereof, wherein said system has a rapid onset of (R)-dihydroetorphine plasma concentration characterised by the mean in vivo plasma concentration of (R)-dihydroetorphine achieving at least 50% of its Cₘₐₓ in less than 20 hours, preferably in less than 18 hours and more preferably in less than 12 hours, after application of the system to the skin of a human subject, e.g. when based on the mean plasma concentration versus time curve.

Viewed from a further aspect, the present invention provides a transdermal delivery system comprising (R)-dihydroetorphine, or a salt, hydrate or a derivative thereof, which, when applied to the skin of a human subject, produces a rapid onset of (R)-dihydroetorphine plasma concentration characterised by the mean in vivo plasma concentration of (R)-dihydroetorphine achieving at least 50% of its Cₘₐₓ in less than 20 hours, preferably in less than 18 hours and more preferably in less than 12 hours, after application of the system, e.g. when based on the mean plasma concentration versus time curve.

Viewed from a further aspect, the present invention provides a system as hereinbefore described for use in medicine.

Viewed from a further aspect, the present invention provides a system as hereinbefore described for use in the treatment of pain.

Viewed from a further aspect, the present invention provides a method for the treatment of pain in a human subject in need thereof comprising applying a system as hereinbefore described to the skin of said human subject.

### DEFINITIONS

As used herein the term “rapid onset” refers to the relatively fast increase in the mean plasma concentration of (R)-dihydroetorphine which occurs after application of a system (e.g. patch) to the skin of a human subject.

As used herein the term “rapid offset” refers to the relatively fast decrease in the mean plasma concentration of (R)-dihydroetorphine which occurs after removeal of a system (e.g. patch) from the skin of a human subject.

As used herein the term Cₘₐₓ refers to the maximum observed plasma concentration of (R)-dihydroetorphine.

As used herein the term AUCt refers to the area under the plasma concentration-time curve measured from the time of dosing to the last measurable concentration.

As used herein the term tₘₐₓ refers to the time to maximum observed plasma concentration.

A number of the pharmacokinetic parameters used herein are defined in terms of values achieved with a single patch having a size of 25 cm² and comprising 6.25 mg of (R)-dihydroetorphine. It is of course intended that this definition also encompasses bioequivalent systems, e.g. patches, thereto.

As used herein the term “transdermal delivery system” refers to a system capable of delivering (R)-dihydroetorphine, or a salt or a hydrate thereof, through the skin or mucosal tissues to the blood stream. A preferred system is a transdermal patch.

As used herein the term “transdermal patch” refers to an adhesive pad capable of delivering (R)-dihydroetorphine, or a salt, or a hydrate or a derivative thereof, through the skin or mucosal tissues to the blood stream and adhering to the skin. The term transdermal patch also encompasses transdermal plaster, transdermal tape and transdermal disc.

As used herein the term “layer” refers to a continuous body or film of material. Layers do not have any breaks or interruptions therein. Layers may or may not have a uniform thickness. Layers may or may not be planar.

As used herein the term “laminate” refers to a multilayered structure comprising at least two layers connected or bonded together. Preferred patches of the present invention are laminates.

As used herein the term “backing layer” refers to a layer that is a constituent of a patch, which in use of the patch, is remote to the skin. The backing layer covers the drug-containing layer and thereby protects it from exposure to the environment.

As used herein the term “drug-containing layer” refers to a layer comprising (R)-dihydroetorphine, or a salt, or a hydrate thereof, and optionally other active ingredients. In use the drug-containing layer is in contact with the skin.

As used herein the term “pressure sensitive adhesive” refers to an adhesive that requires only minimal pressure, e.g. manual pressure, to stick to the surface of the skin.

As used herein the term “release liner” refers to a removable layer of the patch that is removed prior to application of the patch to skin. The purpose of the release liner is to prevent the patch from loss of drug prior to its application to the skin.

As used herein the term “poly(meth)acrylate” refers to a polymer comprising acrylate and/or methacrylate monomers. These polymers are also often referred to as acrylic acid ester and methacrylic acid ester polymers.

The terms pain relief and analgesia are used herein interchangeably.

### DESCRIPTION OF THE INVENTION

The present invention provides a transdermal delivery system (e.g. a patch) which achieves a rapid onset in the plasma concentration of (R)-dihydroetorphine, or a salt or hydrate or derivative thereof, of the human subject to which the system is applied. Thus the mean plasma concentration of (R)-dihydroetorphine in the human subject increases rapidly after application of the system to the skin of the human subject. This is highly advantageous since once a certain threshold level of (R)-dihydroetorphine is achieved, pain relief is provided. Thus pain relief is provided relatively quickly after application of the system of the invention to the skin of the human subject.

The present invention provides a transdermal delivery system (e.g. patch) comprising (R)-dihydroetorphine, or a salt or hydrate thereof, wherein said system has a rapid onset of (R)-dihydroetorphine plasma concentration characterised by the mean in vivo plasma concentration of (R)-dihydroetorphine achieving at least 50% of its Cₘₐₓ in less than 20 hours, preferably in less than 18 hours and more preferably in less than 12 hours, after application of the system to the skin of a human subject, e.g. when based on the mean plasma concentration versus time curve. Expressed alternatively the present invention provides a transdermal delivery system (e.g. patch) comprising (R)-dihydroetorphine, or a salt or a hydrate or a derivative thereof, which, when applied to the skin of a human subject, produces a rapid onset of (R)-dihydroetorphine plasma concentration characterised by the mean in vivo plasma concentration of (R)-dihydroetorphine achieving at least 50% of its Cₘₐₓ in less than 20 hours, preferably in less than 18 hours and more preferably in less than 12 hours, after application of the system (e.g. patch) to the skin of a human subject, e.g. when based on the mean plasma concentration versus time curve. The systems (e.g. patches) may, for example, achieve 50% of its Cₘₐₓ in 4 to 20 hours, more preferably 6 to 18 hours and still more preferably 8 to 12 hours, e.g. when based on the mean plasma concentration versus time curve.

Preferred systems (e.g. patches) of the invention are characterised by the mean in vivo plasma concentration of (R)-dihydroetorphine achieving at least 25% of its Cₘₐₓ in less than 10 hours, preferably in less than 8 hours and more preferably in less than 6 hours, after application of the system (e.g. patch) to the skin of a human subject, e.g. when based on the mean plasma concentration versus time curve. The system (e.g. patch) may, for example, achieve 25% of its Cₘₐₓ in 0.5 to 10 hours, more preferably 0.75 to 8 hours and still more preferably 1 to 6 hours, e.g. when based on the mean plasma concentration versus time curve.

Further preferred systems (e.g. patches) of the invention are characterised by the mean in vivo plasma concentration of (R)-dihydroetorphine achieving at least 75% of its Cₘₐₓ in less than 24 hours, preferably in less than 18 hours and more preferably in less than 16 hours after application of the system (e.g. patch) to the skin of a human subject, e.g. when based on the mean plasma concentration versus time curve. The system (e.g. patch) may, for example, achieve 75% of its Cₘₐₓ in 6 to 24 hours, more preferably 8 to 18 hours and still more preferably 10 to 16 hours, e.g. when based on the mean plasma concentration versus time curve.

Further preferred systems (e.g. patches) of the invention are characterised by the mean in vivo plasma concentration of (R)-dihydroetorphine achieving Cₘₐₓ in less than 36 hours, preferably less than 30 hours and more preferably less than 28 hours after application of the system (e.g. patch) to the skin of a human subject, e.g. when based on the mean plasma concentration versus time curve. Cₘₐₓ may, for example, be achieved in 16 to 36 hours, more preferably 18 to 30 hours and still more preferably 20 to 28 hours, e.g. when based on the mean plasma concentration versus time curve.

Further preferred systems (e.g. patches) of the invention are characterised by the mean in vivo plasma concentration of (R)-dihydroetorphine being at least 10 pg/mL in less than 12 hours, preferably in less than 10 hours and more preferably in less than 8 hours after application of the system (e.g. patch) to the skin of a human subject. The minimum time to achieve a mean plasma concentration of 10 pg/ml may be, for example, 1 hour or less than 1 hour (for example 30 minutes). Yet further preferred systems (e.g. patches) of the invention are characterised by the mean in vivo plasma concentration of (R)-dihydroetorphine being at least 50 pg/mL in less than 14 hours, preferably in less than 12 hours and more preferably in less than 10 hours after application of the system (e.g. patch) to the skin of a human subject. The minimum time to achieve a mean plasma concentration of 50 pg/ml may be, for example, 2 hours, or less than 2 hours (for example 30 minutes). Preferably the system (e.g. patch) which achieves these mean plasma concentrations of (R)-dihydroetorphine is a single patch having a size of 25 cm² and comprising 6.25 mg of (R)-dihydroetorphine.

In a further preferred system (e.g. patch) of the invention, the mean in vivo plasma concentration of (R)-dihydroetorphine increases at an average rate of 5 to 20 pg/ml/h until the mean in vivo concentration of (R)-dihydroetorphine reaches 50% of Cₘₐₓ (e.g. when based on the mean plasma concentration versus time curve) and preferably when a single patch having a size of 25 cm² and comprising 6.25 mg of (R)-dihydroetorphine is applied to the skin of a human subject.

In a further preferred system (e.g. patch) of the invention the mean in vivo plasma concentration of (R)-dihydroetorphine is 80 to 125% of 50 pg/ml in less than 8 hours, preferably in less than 7 hours and more preferably in less than 6 hours after application of the system (e.g. patch) to the skin of a human subject, e.g. when a single patch having a size of 25 cm² and comprising 6.25 mg of (R)-dihydroetorphine is applied. Preferably the mean in vivo plasma concentration of (R)-dihydroetorphine is 80 to 125% of 50 pg/ml in 0.25 to 8 hours, more preferably 0.5 to 7 hours and still more preferably 0.75 to 6 hours, e.g. when a single patch having a size of 25 cm² and comprising 6.25 mg of (R)-dihydroetorphine is applied to the skin of a human subject .

In a further preferred system (e.g. patch) of the invention the mean in vivo plasma concentration of (R)-dihydroetorphine is 80 to 125% of 100 pg/ml in less than 12 hours, preferably in less than 11 hours and more preferably in less than 10 hours after application of the system (e.g. patch) to the skin of a human subject, e.g. when a single patch having a size of 25 cm² and comprising 6.25 mg of (R)-dihydroetorphine is applied. Preferably the mean in vivo plasma concentration of (R)-dihydroetorphine is 80 to 125% of 100 pg/ml in 0.5 to 12 hours, more preferably 0.75 to 11 hours and still more preferably 1 to 10 hours, e.g. when a single patch having a size of 25 cm² and comprising 6.25 mg of (R)-dihydroetorphine is applied to the skin of a human subject.

In a further preferred system (e.g. patch) of the invention the mean in vivo plasma concentration of (R)-dihydroetorphine is 80 to 125% of 10 pg/ml in less than 6 hours, preferably in less than 5 hours and more preferably in less than 4 hours, after application of the system to the skin of a human subject, e.g. when a single patch having a size of 25 cm² and comprising 6.25 mg of (R)-dihydroetorphine is applied. Preferably the mean in vivo plasma concentration of (R)-dihydroetorphine is 80 to 125% of 10 pg/ml in 10 minutes to 6 hours, more preferably 15 minutes to 5 hours and still more preferably 20 minuties to 4 hours, e.g. when a single patch having a size of 25 cm² and comprising 6.25 mg of (R)-dihydroetorphine is applied to the skin of a human subject.

Preferably the transdermal delivery system (e.g. patch) of the present invention achieves a rapid offset in the plasma concentration of (R)-dihydroetorphine, or a salt or hydrate thereof, in the plasma of the human subject from which the system is removed. Thus the mean plasma concentration of (R)-dihydroetorphine in the subject decreases rapidly after removal of the system (e.g. patch) from the skin of the human subject. This is highly advantageous since it means, for example, that a different treatment regime or course of treatment can commence more quickly thereafter. In preferred systems (e.g. patches) of the invention, the system (e.g. patch) has a rapid offset in (R)-dihydroetorphine plasma concentration characterised by the mean in vivo plasma concentration of (R)-dihydroetorphine reducing from its concentration at the time of removal of the system from the skin of the human subject by at least 50% in less than 16 hours, preferably in less than 14 hours and more preferably in less than 12 hours. Preferably the mean in vivo plasma concentration of (R)-dihydroetorphine reduces from its concentration at the time of removal of the system (e.g. patch) from the skin of the human subject by at least 50% in 4 to 16 hours, more preferably 6 to 14 hours and still more preferably 8 to 12 hours.

In further preferred systems (e.g. patches) of the invention, the system has a rapid offset in (R)-dihydroetorphine plasma concentration characterised by the mean in vivo plasma concentration of (R)-dihydroetorphine reducing from its concentration at the time of removal of the system from the skin of the human subject by at least 25% in less than 8 hours, preferably in less than 6 hours and more preferably in less than 4 hours. Preferably the mean in vivo plasma concentration of (R)-dihydroetorphine reduces from its concentration at the time of removal of the system from the skin of the human subject by at least 25% in 1 to 8 hours, more preferably 2 to 6 hours and still more preferably 2 to 4 hours.

In further preferred systems (e.g. patches) of the invention, the mean in vivo plasma concentration of (R)-dihydroetorphine is less than 50 pg/ml in less than 12 hours, preferably less than 10 hours and more preferably in less than 8 hours, after removal of the system (e.g. patch) from the skin of the human subject. Preferably the mean in vivo plasma concentration of (R)-dihydroetorphine is less than 50 pg/ml in 0.5 to 12 hours, more preferably 1 to 10 hours and still more preferably 2 to 8 hours. In further preferred systems of the invention, the mean in vivo plasma concentration of (R)-dihydroetorphine is less than 10 pg/ml in less than 48 hours, preferably less than 36 hours and more preferably in less than 24 hours, after removal of the system (e.g. patch) from the skin of the human subject. Preferably the mean in vivo plasma concentration of (R)-dihydroetorphine is less than 10 pg/ml in 8 to 48 hours, more preferably 10 to 36 hours and still more preferably 12 to 24 hours. Preferably the system (e.g. patch) which achieves these mean plasma concentrations of (R)-dihydroetorphine is a single patch having a size of 25 cm² and comprising 6.25 mg of (R)-dihydroetorphine.

In a further preferred system (e.g. patch) of the invention, the mean in vivo plasma concentration of (R)-dihydroetorphine is 80 to 125% of 80 pg/ml in less than 10 hours, preferably in less than 8 hours and more preferably in less than 6 hours after removal of the system (e.g. patch) from the skin of the human subject, e.g. when a single patch having a size of 25 cm² and comprising 6.25 mg of (R)-dihydroetorphine is applied. Preferably the mean in vivo plasma concentration of (R)-dihydroetorphine is 80 to 125% of 80 pg/ml in 0.5 to 10 hours, more preferably 0.75 to 8 hours and still more preferably 1 to 6 hours, e.g. when a single patch having a size of 25 cm² and comprising 6.25 mg of (R)-dihydroetorphine is applied to the skin of a human subject.

In further preferred systems (e.g. patches) of the invention, the mean in vivo plasma concentration of (R)-dihydroetorphine is 80 to 125% of 50 pg/ml in less than 12 hours, preferably in less than 10 hours and more preferably in less than 8 hours after removal of the system from the skin of the human subject, e.g. when a single patch having a size of 25 cm² and comprising 6.25 mg of (R)-dihydroetorphine is applied. Preferably the mean in vivo plasma concentration of (R)-dihydroetorphine is 80 to 125% of 50 pg/ml in 0.75 to 12 hours, more preferably 1 to 10 hours and still more preferably 1.5 to 8 hours, e.g. when a single patch having a size of 25 cm² and comprising 6.25 mg of (R)-dihydroetorphine is applied to the skin of the human subject.

In further preferred systems of the invention, the mean in vivo plasma concentration of (R)-dihydroetorphine is 80 to 125% of 40 pg/ml in less than 12 hours, preferably in less than 10 hours and more preferably in less than 8 hours after removal of the system from the skin of the human subject, e.g. when a single patch having a size of 25 cm² and comprising 6.25 mg of (R)-dihydroetorphine is applied. Preferably the mean in vivo plasma concentration of (R)-dihydroetorphine is 80 to 125% of 40 pg/ml in 1 to 12 hours, more preferably 1.5 to 10 hours and still more preferably 2 to 8 hours, e.g. when a single patch having a size of 25 cm² and comprising 6.25 mg of (R)-dihydroetorphine is applied to the skin of the human subject.

The transdermal delivery systems (e.g. patches) of the present invention preferably maintain a relatively high mean plasma concentration of (R)-dihydroetorphine for an extended period of time. This is advantageous because it means that pain relief may be provided for an extended period of time, e.g. for up to 168 hours. Thus in further preferred systems of the invention, the mean in vivo plasma concentration of (R)-dihydroetorphine is at least 50% of Cₘₐₓ for at least 72 hours, preferably at least 84 hours and more preferably at least 96 hours after Cₘₐₓ is achieved, e.g. when based on the mean plasma concentration versus time curve. Preferably the mean in vivo plasma concentration of (R)-dihydroetorphine is at least 50% of Cₘₐₓ for 72 to 168 hours, more preferably 84 to 156 hours and still more preferably 96 to 144 hours, e.g. when based on the mean plasma concentration versus time curve.

In further preferred systems (e.g. patches) of the invention, the mean in vivo plasma concentration of (R)-dihydroetorphine is at least 40% of Cₘₐₓ for at least 96 hours, preferably at least 108 hours and more preferably at least 125 hours after application of the system (e.g. patch) to the skin of the human subject, e.g. when based on the mean plasma concentration versus time curve. Preferably the mean in vivo plasma concentration of (R)-dihydroetorphine is at least 40% of Cₘₐₓ for 96 to 168 hours, more preferably 108 to 156 hours and still more preferably 120 to 156 hours, e.g. when based on the mean plasma concentration versus time curve.

In further preferred systems (e.g. patches) of the invention, the mean in vivo plasma concentration of (R)-dihydroetorphine is at least 25% of Cₘₐₓ for at least 144 hours, more preferably at least 156 hours and still more preferably at least 168 hours after application of the system (e.g. patch) to the skin of the human subject, e.g. when based on the mean plasma concentration versus time curve. Preferably the mean in vivo plasma concentration of (R)-dihydroetorphine is at least 25% of Cₘₐₓ for 144 to 216 hours, more preferably 156 to 204 hours and still more preferably 168 to 192 hours, e.g. when based on the mean plasma concentration versus time curve.

In a further preferred system (e.g. patch) of the invention, the mean in vivo plasma concentration of (R)-dihydroetorphine is at least 50 pg/ml for at least 72 hours, preferably at least 84 hours and more preferably at least 96 hours after Cₘₐₓ is achieved (e.g. when based on the mean plasma concentration versus time curve) and preferably when a single patch having a size of 25 cm² and comprising 6.25 mg of (R)-dihydroetorphine is applied to the skin of the human subject. Preferably the mean in vivo plasma concentration of (R)-dihydroetorphine is at least 50 pg/ml for 72 to 168 hours, more preferably 84 to 156 hours and still more preferably 96 to 144 hours (e.g. when based on the mean plasma concentration versus time curve) e.g. when a single patch having a size of 25 cm² and comprising 6.25 mg of (R)-dihydroetorphine is applied to the skin of the human subject.

Preferred systems (e.g. patches) of the invention achieve a dose adjusted Cₘₐₓ of 80 to 125% of about 200 pg/ml, relative to a single patch having a size of 25 cm² and comprising 6.25 mg of (R)-dihydroetorphine, e.g. when based on the mean plasma concentration versus time curve. This means, for example, that two patches of the same size, each comprising 6.25 mg of (R)-dihydroetorphine, achieve a Cₘₐₓ of approximately 400 pg/ml (e.g.about 360 pg/ml), i.e. two times 200 pg/ml.

Other preferred systems (e.g. patches) of the invention achieve a dose adjusted AUCt of 80 to 125% of 16210 pg.h/ml, relative to a single patch having a size of 25 cm² and comprising 6.25 mg of (R)-dihydroetorphine e.g. when based on the mean plasma concentration versus time curve. This means, for example, that a ½ patch of the same size and comprising 3.125 mg of (R)-dihydroetorphine, achieves a AUCt of approximately 8105 pg.h/ml.

Other preferred systems (e.g. patch) of the invention have a mean tₘₐₓ of 30 to 70 hours and more preferably 35 to 50 hours.

Yet further preferred systems (e.g. patches) of the invention achieve a mean in vivo flux rate of (R)-dihydroetorphine of 5 to 15 pg/h, more preferably 6 to 12 pg/h and still more preferably 7 to 10 pg/h, during a period of 168 hours, when a single patch having a size of 25 cm² and comprising 6.25 mg of (R)-dihydroetorphine is applied to the skin of the human subject.

The transdermal system (e.g. patch) of the present invention preferably comprises a drug-containing layer comprising (R)-dihydroetorphine, or a salt or a hydrate thereof, and a poly(meth)acrylate; and a backing layer. In use, the drug-containing layer is in contact with the skin and the backing layer is remote to the skin.

Preferred transdermal systems (e.g. patches) of the present invention further comprise a release liner which is removable or detachable. When present, the release liner is present on the opposite side of the drug-containing layer to the backing layer. The release liner is removed or detached prior to use of the transdermal system (e.g. patch) to expose a surface of the drug-containing layer for contact with the skin. Preferred transdermal systems (e.g. patches) of the present invention are self-adhering. Thus when the release liner is removed and the system (e.g. patch) is applied to the patient's skin, the patch remains attached thereto without there being a need for any separate attachment mechanism, e.g. straps or tiers.

The transdermal system (e.g. patch) of the present invention may be a drug in adhesive patch or a matrix patch. Preferably the transdermal patch is a drug in adhesive patch, such as a single layer or multi-layer drug in adhesive patch. Single layer drug in adhesive patches are most preferred. Preferably the drug in adhesive layer is continuous. Particularly preferably the drug in adhesive layer does not comprise any channels.The transdermal system (e.g. patch) of the present invention may comprise 2, 3, 4 or 5 layers. Preferred systems (e.g. patches) comprise 3 or 5 layers and especially preferably 3 layers.

Preferred transdermal systems (e.g. patches) of the present invention have the structures A, B, C or D comprising (e.g. consisting of) the following layers, wherein the layers are present in the numerical order specified:

(A) (i) a backing layer;
(ii) a drug-containing layer comprising (R)-dihydroetorphine, or a salt or a hydrate thereof, and a poly(meth)acrylate; and
(iii) optionally a release liner.

(B) (i) a backing layer;
(ii) a first drug-containing layer comprising (R)-dihydroetorphine, or a salt or a hydrate thereof, and a poly(meth)acrylate;
(iii) a separating layer;
(iv) a second drug-containing layer comprising a drug; and
(v) optionally a release liner.

(C) (i) a backing layer;
(ii) an adhesive layer;
(iii) a separating layer;
(iv) a drug-containing layer comprising (R)-dihydroetorphine, or a salt or a hydrate thereof, and a poly(meth)acrylate; and
(v) optionally a release liner.

(D) (i) a backing layer;
(ii) a drug-containing layer comprising (R)-dihydroetorphine, or a salt or a hydrate thereof and a poly(meth)acrylate;
(iii) a separating layer;
(iv) an adhesive layer; and
(v) optionally a release liner.

In transdermal systems (e.g. patches) having the structure (A), (B) or (D), each of the layers is preferably planar. In transdermal systems (e.g. patches) having the structure (C), the backing layer, the separating layer, the drug-containing layer and, when present, the release liner are preferably planar. The adhesive layer present in structure (C) is preferably non-planar. Preferably the adhesive layer, together with the release liner, surrounds the separating layer and the drug-containing layer, i.e. the separating layer and the drug-containing layer are encapsulated or encompassed.

Particularly preferred transdermal systems (e.g. patches) of the present invention are those having the structures (A), (B) or (C), more preferably (A) or (C) and still more preferably (A). Preferred transdermal systems (e.g. patches) comprise a release liner. Preferred transdermal patches do not comprise an adverse agent layer.

The drug-containing layer of the transdermal system (e.g. patch) of the present invention comprises (R)-dihydroetorphine. The (R)-dihydroetorphine may be present in the form of a free base or a pharmaceutically acceptable salt. Whether present as a free base or as a pharmaceutically acceptable salt, the (R)-dihydroetorphine may be present in anhydrous form or in the form of a hydrate.

Preferred salts are those that retain the biological effectiveness and properties of (R)-dihydroetorphine and are formed from suitable non-toxic organic or inorganic acids. Acid addition salts are preferred. Representative examples of salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid and trifluoro acetic acid. The modification of a compound into a salt is a technique well known to chemists to obtain improved physical and chemical stability, hygroscopicity, flowability and solubility of compounds.

Particularly preferably the drug-containing layer comprises (R)-dihydroetorphine in the form of free base.

The drug-containing layer of the transdermal system (e.g. patch) of the present invention may comprise (R)-dihydroetorphine, or a salt, or a hydrate thereof, as the sole active ingredient. Alternatively (R)-dihydroetorphine, or a salt, or a hydrate thereof, may be present in combination with another active ingredient. More preferably, however, (R)-dihydroetorphine, or a salt, or a hydrate thereof, is the sole active ingredient present in the drug-containing layer. Still more preferably (R)-dihydroetorphine, or a salt, or a hydrate thereof, is the sole active ingredient present in the system (e.g. patch). Particularly preferably the patch does not comprise an adverse agent.

The drug-containing layer preferably comprises an adhesive and more preferably a pressure sensitive adhesive. The presence of a pressure sensitive adhesive enables the system (e.g. patch) to adhere to the skin of a patient. In preferred systems (e.g. patches) of the present invention no adhesive layer that is separate to the drug-containing layer is required. Instead the adhesive and drug are preferably both incorporated into the drug-containing layer. This simplifies the design and optimisation of the system (e.g. patch).

In a preferred embodiment of the present invention the drug-containing layer comprises a poly(meth)acrylate. The poly(meth)acrylate may be an adhesive and/or a matrix polymer. Preferably the poly(meth)acrylate is an adhesive.

Preferably the poly(meth)acrylate is a copolymer. Preferred copolymers comprise at least two alkyl (meth)acrylate monomers. For example, the copolymer may comprise at least two alkyl acrylate monomers, at least two alkyl methacrylate monomers or may comprise at least one alkyl acrylate monomer and at least one alkyl methacrylate monomer.

In preferred poly(meth)acrylates present in the drug-containing layer of the present invention the alkyl (meth)acrylate monomers comprise 1 to 12 carbon atoms in the alkyl group. Preferably the alkyl (meth)acrylate monomers are selected from methyl acrylate, ethyl acrylate, propyl acrylate, butyl acrylate, isobutyl acrylate, pentyl acrylate, hexyl acrylate, 2-ethylhexyl acrylate, octyl acrylate, isooctyl acrylate, decyl acrylate, dodecyl acrylate, methyl methacrylate, ethyl methacrylate, propyl methacrylate, butyl methacrylate, isobutyl methacrylate, pentyl methacrylate, hexyl methacrylate, 2-ethylhexyl methacrylate, octyl methacrylate, isooctyl methacrylate, decyl methacrylate, dodecyl methacrylate and isomers thereof.

The poly(meth)acrylate may further comprise other monomers. The poly(meth)acrylate may, for example, comprise one or more vinyl ester monomers, e.g. vinyl acetate. Preferably, however, the poly(meth)acrylate does not comprise vinyl ester monomers.

The poly(meth)acrylate may further comprise one or more functionalised monomers. Preferred functionalised monomers are carboxy and hydroxy funtionalised monomers. Preferred carboxy functionalised monomers comprise 3 to 6 carbon atoms. Representative examples of suitable carboxy functionalised monomers include acrylic acid, methacrylic acid, methacrylic acid, itaconic acid, maleic acid, maleic anhydride, and beta-carboxyethyl acrylate. Representative examples of suitable hydroxy functionalised monomers include hydroxyethyl acrylate, hydroxypropyl acrylate, hydroxyethyl methacrylate and hydroxypropyl methacrylate. Preferably, however, the poly(meth)acrylate does not comprise functionalised, e.g. carboxy or hydroxy, functionalised monomers.

The poly(meth)acrylate may further comprise crosslinkable monomers. Representative examples of suitable monomers include glycidyl methacrylate, allyl glycidyl ether and hexanedioldi(methy)acrylate. Preferably, however, the poly(meth)acrylate does not comprise crosslinkable monomers.

The poly(meth)acrylate may further comprise a nitrogen-containing monomer and preferably a N-substituted acryamide or methacrylamide monomer. Representative examples of suitable monomers include N-vinyl pyrrolidine, N-vinyl caprolactam, N-tertiary octyl acrylamide, dimethyl acrylamide, diacetone acrylamide, N-tertiary butyl acryamide, N-isopropyl acrylamide, N-vinyl acetamide and/or N-vinyl formamide. The poly(meth)acrylate may further comprise an amine-containing monomer, e.g. 2-(diethylamino)ethyl methacrylate. Amine-containing monomers impart functionality to the adhesive. Preferably, however, the poly(meth)acrylate does not comprise nitorgen-containing monomers.

Other comonomers that may be present in the poly(meth)acrylate include styrene and nitriles, e.g. acrylonitrile and cyanoethylacrylate. Such comonomers may be incorporated into the polymer to control its glass transition temperature. Preferably, however, the poly(meth)acrylate does not comprise styrene or nitrile monomers.

Preferred poly(meth)acrylate present in the drug-containing layer comprises 40-100% mol of alkyl acrylate monomers and alkyl methacrylate monomers and 0 to 60% mol of another monomer, more preferably 70-100% mol of alkyl acrylate and alkyl methacrylate monomers and 0 to 30% mol of another monomer and still more preferably 90-100% mol of alkyl acrylate and alkyl methacrylate monomers and 0 to 10% mol of another monomer. Still more preferably the poly(meth)acrylate consists of alkyl acrylate monomers and/or alkyl methacrylate monomers. It has been found that this produces the most stable systems (e.g. patches).

Suitable alkyl acrylate and/or alkyl methacrylate copolymers for use in the present invention are commercially available from Henkel under the trade name Duro-Tak. These include, for example: Duro-Tak 87-900A, 87-9301, 87-4098 and 87-9088, acryate polymers which are supplied in an organic solvent (ethyl acetate) and have no hydroxy or carboxyl functional groups; Duro-Tak 87-202A and 387-2510/87-2510, acrylate polymers which are supplied in an organic solvent (ethyl acetate) all having —OH functional groups; Duro-Tak 87-208A, 387-2287/87-2287 and 87-4287 acrylate-vinyl acetate polymers which are supplied in an organic solvent (ethyl acetate) solution all having -OH functional groups; and Duro-Tak 387-2516/87-2516 and 387-2525/87-2525 acrylate-vinyl acetate polymers supplied in an organic solvent solution all having —OH functional groups. Particularly preferred copolymers are listed in the table below.

| Tradename | Monomers | Characteristics |
|---|---|---|
| Duro-Tak 87-9301 | Alkyl acrylates; no other | No OH or COOH |
| | monomers | functional groups |
| Duro-Tak 87-2510 | Alkyl acrylates and | OH functional groups |
| | hydroxy-containing | present |
| | monomer | |
| Duro-Tak 87-503A | Acrylic-rubber hybrid | |
| Duro-Tak 87-202A | Alkyl acrylates and | OH functional groups |
| | hydroxy-container | present |
| | monomer | |

When mixed with a poly(meth)acrylate in the drug-containing layer, (R)-dihydroetorphine, or a salt, or a hydrate thereof, shows remarkable physical stability, and significantly improved stability compared to drug-containg layers comprising styrene-isobutylene-styrene and polyisobutylene. Thus when present with poly(meth)acrylate, the (R)-dihydroetorphine, or a salt, or a hydrate thereof, shows no tendency to crystallise, even under extreme forced conditions. This is particularly the case when the poly(meth)acrylate consists of alkyl acrylate monomers and/or alkyl methacrylate monomers.

The drug-containing layer of the present invention optionally comprises a second polymer. Representative examples of other polymers include silicone polymers such as polydimethylsiloxane and polymethylphenylsiloxane and rubber polymers such as polyisobutylene and styrene-isoprene-styrene block copolymer. Preferably, however, poly(meth)acrylate is the sole polymer present in the drug-containing layer. This is advantageous as it yields systems (e.g patches) having the longest storage capabilities.

The drug-containing layer of the present invention may further comprise a permeation enhancer. Thus in some embodiments the drug-containing layer further comprises a skin permeation enhancer. The permeation enhancer is preferably a C₁₋₂₀ monohydric or polyhydric alcohol, C₂₋₂₀ fatty acid, esters of C₂₋₂₀ fatty acid acids and C₁₋₂₀ monohydric or polyhydric alcohols, urea, pyrrolidine derivative, cyclic monoterpenes, 1-dodecylazacycloheptane-2-one, cyclodextrin or calcium thioglycolate.

Representative examples of permeation enhancers include methyl alcohol, ethyl alcohol, propyl alcohol, isopropyl alcohol, butyl alcohol, heptyl alcohol, octyl alcohol, capryl alcohol, nonyl alcohol, decyl alcohol, undecyl alcohol, lauryl alcohol, tridecyl alcohol, myristyl alcohol, pentadecyl alcohol, cetyl alcohol, hexadecyl alcohol, heptadecyl alcohol, stearyl alcohol, oleyl alcohol, nonadecyl alcohol, eicosyl alcohol, ethylene glycol, propylene glycol, 1,3 butadiol, glycerin, acetic acid, propionic acid, butyric acid, valeric acid, caproic acid, enanthic acid, caprylic acid, pelagonic acid, capric acid, lauric acid, myristic acid, palmitic acid, stearic acid, benzoic acid, salicylic acid, lactic acid, oxalic acid, malonic acid, succinic acid, glutaric acid, adipic acid, maleic acid, fumaric acid, malic acid, tartaric acid, phthalic acid, myristyl lactate, cetyl lactate, lauryl lactate, isopropyl myristate, isopropyl palmitate, butyl stearate, myristyl myristate, urea, thiourea, 2-pyrrolidone, 1-methyl-2-pyrrolidone, 5-methyl-2-pyrrolidone, 1,5-dimethyl pyrrolidone, 1-ethyl pyrrolidone, menthol, limonene and a-terpenol. Yet further examples of permeation enhancers include oleic acid, triacetin, levulinic acid, dodecanol and lauryl acetate.

Preferably the permeation enhancer is selected from oleic acid, oleyl alcohol, triacetin, levulinic acid, dodecanol and lauryl acetate. Particularly preferably the permeation enhancer is selected from oleic acid, oleyl alcohol and triacetin. These enhancers have been found to increase the flux of (R)-dihydroetorphine, or a salt, or a hydrate thereof, and also to provide systems (e.g. patches) that are stable, even under forced conditions. A mixture of more than one permeation enhancer may be used. For example, a mixture of two or more of the following may be used: oleic acid, oleyl alcohol, triacetin, levulinic acid, dodecanol and lauryl lactate. In a particular embiodiment when a mixture is used, two or more enhancers selected form oleic acid, oleyl alcohol and triacetin are used.

In more preferred embodiments the drug-containing layer does not comprise a permeation enhancer.

The drug-containing layer may optionally comprise a permeation-sustaining agent. Preferably the permeation sustaining agent is a C₁₂₋₃₂ hydrocarbon, C₁₂₋₃₂ alcohol, glycol, C₆₋₃₂ fatty acid, C₆₋₃₂ fatty acid ester, vegetable oil, animal oil, rubber, polyurethane, silicone resin, water-soluble polymer compound, cellulose, urea, cyclodextrin, thickening agent, clay, gelling agent, suspending agent and emulsifying agent.

Representative examples of permeation-sustaining agents include liquid paraffin, which is a mixture of various hydrocarbons, branched-chain paraffins, solid paraffin, white Vaseline, lauryl alcohol, tridecyl alcohol, myristyl alcohol, pentadecyl alcohol, cetyl alcohol, hexadecyl alcohol, heptadecyl alcohol, steryl alcohol, oleyl alcohol, nonadecyl alcohol, eicosyl alcohol, seryl alcohol, melissyl alcohol, ethylene glycol, propylene glycol, trimethylene glycol, 1,3-butane diol, polyethylene glycol and mixtures obtained by mixing in a suitable ratio polyethylene glycols of a low degree of polymerisation such as Macrogol 400 (trade name) and polyethylene glycols of a high degree of polymerisation, such as Macrogol 4000 (trade name), caproic acid, enanthic acid, caprylic acid, pelargonic acid, capric acid, undecyl acid, lauric acid, tridecyl acid, myristic acid, pentadecyl acid, palmitic acid, heptadecyl acid, stearic acid, oleic acid, nonadecanoic acid, arachidonic acid, linoleic acid, linolenic acid, behenic acid, lignoceric acid, cerotic acid, heptacosanoic acid, montanoic acid, melissic acid, lacceric acid, elaidic acid, brassidic acid, myristyl palmitate, myristyl stearate, myristyl myristate, seryl lignocerate, lacceryl cerotate, lacceryl laccerate, natural waxes of animal origin (e.g. beeswax, whale wax or ceramic wax), vegetable-derived natural waxes (e.g. carnauba wax, candelilla wax), glyceryl monolaurate, glyceryl monomyristearate, glyceryl monostearate, glyceryl mono-oleate, glyceryl dilaurate, glyceryl dimyristate, glyceryl distearate, glyceryl tristearate, glyceryl trimyristae, glyceryl tristearate, castor oil, olive oil, soya oil, sesame oil, almond oil, safflower oil, cottonseed oils, turpentine, hydrogenated vegetable oils, mink oil, egg yolk oil, squalane, squalene, lanolin derivatives, natural rubber, SBS butyl rubber, polyisobutylene, polyvinyl alcohol ether, polyurethane, polyimide, ethylene-vinyl acetate copolymer, dimethyl polysiloxane, polyisoprene rubber, styrene-isoprene-styrene block copolymer, styrene butadiene rubber, polyisobutylene, butylene rubber, polyacrylic acid or salts thereof, acrylic acid ester-acrylic acid copolymer, poly-vinyl alcohol, polyvinyl pyridine, hydroxypropyl cellulose and cross-linked versions thereof, sodium alginate, Arabia gum, pectin, tragacanth gum, ethyl cellulose, hydroxymethyl cellulose, hydroxyethyl starch, bentonite and Veegum HV.

Preferably, however, the drug-containing layer does not comprise a permeation sustaining agent. Particularly preferably the drug-containing layer does not comprise a permeation sustaining agent as described above. This is an advantage of the system (e.g. patch) of the present invention. It minimises compatibility issues between components of the system (e.g. patch)and simplifies its design and optimisation.

The drug-containing layer of the present invention may further comprise other conventional excipients, e.g. tackifiers, pH regulators, fillers, softeners, antioxidants, and viscosity modifying agents. Such additional excipients are preferably added in an amount of less than 30% wt, more preferably less than 20% wt and even more preferably less than 10% wt based on the total weight of the drug-containing layer.

If the adhesive present in the drug-containing layer does not exhibit its adhesive property in the temperature range at which the system is to be applied, a tackifier is preferably added. Suitable tackifiers include terpene-based resins or petroleum-based resins such as alicyclic saturated hydrocarbon resins. The softening point of the tackifier is preferably 60-160° C. Preferably, however, the drug-containing layer does not comprise a tackifier.

The pH of the drug-containing layer is preferably in the range of 6-8 and more preferably 7-7.8. When the pH of the drug-containing layer is below 6, percutaneous absorption of (R)-dihydroetorphine, or a salt, or a hydrate thereof, will tend to be reduced. When the pH of the drug-containing layer is higher than 8, the risk of skin irritation will tend to increase. The pH of the drug-containing layer may be measured, for example, by placing a sample of the system (e.g. patch) with the removable release liner removed, having an actual area of 3.48 cm², in a 20 ml vial and adding 20 ml of purified water to the vial, agitating the vial for 3 days at 150 rpm and using a pH Meter for measurement of the obtained liquid. If the pH is outside of the above range, it may be modified using a pH regulator. Suitable pH regulators include organic or inorganic acids, an organic or inorganic acid metal salt, a metal hydroxide and a metal oxide. Alkali metals and alkaline earth metals may be used as metals for organic or inorganic acid salts. Some specific examples of pH regulators are sodium lactate, sodium acetate, sodium hydroxide, or a combination of an acetic acid salt and acetic acid. Preferably, however, the drug-containing layer does not comprise a pH regulator.

Examples of suitable fillers that may be included in the drug-containing layer of the present invention include colloidal silicon dioxide, bentonite and lactose. Preferably, however, the drug-containing layer does not comprise a filler.

A softener may be included in the drug-containing layer. Representative examples of suitable softeners include liquid paraffin, liquid polybutene, liquid isoprene, squalane and squalene or polar oils including vegetable oils (for example, hydrogenated castor oil, cottonseed oil, palm oil and coconut oil). Preferably, however, the drug-containing layer does not comprise a softener.

An antioxidant may be present in the drug-containing layer to minimise the degradation of (R)-dihydroetorphine, or a salt, or a hydrate thereof, and/or the adhesive. Conventional antioxidants may be employed, e.g. tocopherols, butylated hydroxyanisole, ascorbyl palmitate and ascorbyl stearate. Preferably, however, the drug-containing layer does not comprise an antioxidant.

Examples of suitable viscosity modifying agents that may be present in the drug-containing layer include cellulose derivatives and natural or synthetic gums, such as guar gum and tragacanth. Preferably, however, the drug-containing layer does not comprise viscosity modifying agents.

In preferred systems (e.g. patches) of the invention the drug-containing layer is non-aqueous, i.e. contains essentially no water. Preferably the water content of the drug-containing layer does not exceed 10% based on the total weight of the drug-containing layer.

Particularly preferably, the drug-containing layer consists of (R)-dihyroetorphine, poly(meth)acrylate and optionally a permeation enhancer.

Preferably the drug-containing layer comprises 1 to 10% wt, and more preferably 3 to 7.5% wt, and still more preferably 4 to 6% wt, dihydroetorphine or salt or hydrate thereof, based on the dry weight of the constituents of the drug-containing layer. Preferably the drug-containing layer comprises 70 to 99% wt poly(meth)acrylate, more preferably 90 to 97.5% wt, and still more preferably 92.5 to 95.5% wt, based on the dry weight of the constituents of the drug-containing layer. Preferably the drug-containing layer comprises 0 to 15% wt and more preferably 5 to 10% wt of a permeation enhancer, based on the dry weight of the constituents of the drug-containing layer.

The backing layer is preferably impermeable to (R)-dihydroetorphine, or a salt, or a hydrate thereof, and any other active agent present in the system (e.g. patch). Preferably the backing layer is occlusive. The backing layer preferably serves as a protective cover and may also provide a support function. Preferably the backing layer is flexible so that it can accommodate movement of the patient without breaking. The backing layer is preferably applied to one side of the drug-containing layer.

The backing layer may be formed from a range of different materials including film, fabric, foamed sheet, microporous sheet, textile fabrics, foil or a laminate of the afore-going. Preferably, however, the backing layer is a film, e.g. a polymer fillm. Particularly preferred backing layers comprise a polyolefin (e.g. high and low density polyethylene, polypropylene), fluoropolymer (e.g. polytetrafluoroethylene), nylon, cellulose derivatives, ethylene-vinyl acetate, vinyl acetate, polyvinylchloride, polyurethane, polyesters (e.g. polyethylene phthalate, polyethylene terephthalate, polybutylene terephthalate or polyethylene naphthalate), metal foils (e.g. aluminium) and laminates of the afore-going.

Preferred backing layers are laminates. Laminates are generally preferred since it is possible to combine materials having different properties to provide laminates having an attractive balance of properties. Particularly preferred laminates comprise a polyolefin, a polyester and a metal.

Suitable backing layers are commercially available from a range of suppliers, e.g. 3M. Scotchpak 9738 is an example of a preferred backing layer.

Preferred systems (e.g. patches) of the present invention also comprise a removable release liner. The removable release liner is removed prior to application of the system (e.g. patch) to a human subject, e.g. a patient. The removable layer is preferably applied to the opposite side of the drug-containing layer to the backing layer.

The release liner preferably comprises polyolefin (e.g. high and low density polyethylene, polypropylene), fluoropolymer (e.g. polytetrafluoroethylene), nylon, cellulose derivatives, ethylene-vinyl acetate, vinyl acetate, polyvinylchloride, polyurethane, polyesters (e.g. polyethylene phthalate, polyethylene terephthalate, polybutylene terephthalate or polyethylene naphthalate) and laminates of the afore-going. Preferably the release liner comprises silicone, fluropolymer or a mixture thereof.

Some preferred release liners comprise polyesters, particularly polyethylene terephthalate. Other preferred release liners comprise a silicone and/or fluoropolymer (e.g. Teflon) coating, particularly preferably on the side of the release liner contacting the drug containing layer. The coating may, for example, be provided on a release liner as described above. The silicone or fluoropolymer coating enables the release liner to be easily removed without damaging the drug-containing layer to which it is attached.

Suitable release liners are commercially available form a range of suppliers, e.g. Loparex and 3M. Loparex Primeliner FL 2000 and Scotchpak 1022 release liners are examples of preferred release liners.

When a separate adhesive layer is present, it preferably comprises a pressure sensitive adhesive. Preferred pressure sensitive adhesives are selected from styrene-based block copolymers, polyvinyl acetates, poly(iso)butylenes, natural and synthetic rubbers, polyurethanes, polyisoprenes, organopolysiloxanes and poly(meth)acrylates. Still more preferably the pressure sensitive adhesive is selected from styrene-based block copolymers, polyisobutylenes, organopolysiloxanes and poly(meth)acrylates and yet more preferably organopolysiloxanes and poly(meth)acrylates. Poly(meth)acrylates are especially preferred. Preferably the same adhesive is present in this layer as in the drug-containing layer.

Representative examples of styrene-based block copolymers include styrene-isoprene-styrene block copolymer, styrene-butadiene-styrene block copolymer, styrene-ethylene/butylene-block copolymer and styrene-isobutylene-styrene block copolymer. Styrene-isobutylene-styrene block copolymers are particularly preferred. Suitable styrene-based block copolymers are commercially available, e.g. from Henkel. Duro Tak 87-6911 is an example of a suitable styrene-based block copolymer.

Polybutylenes may comprise polybutylene and/or polyisobutylene. Polyisobutylenes are preferred. Suitable polyisobutylene polymers are commercially available, e.g. from Henkel. Duro Tak 87-618A is an example of a suitable polyisobutylene.

Organopolysiloxanes that are suitable for use in the present invention include polydimethylsiloxanes and polydimethyldiphenylsiloxanes. Suitable organopolysiloxanes are commercially available from Dow Corning Corporation under the tradename BIO-PSA. BIO-PSA 7-4302 is particularly preferred.

Preferred poly(meth)acrylates are those described above in relation to the drug-containing layer.

When present the separating layer preferably comprises a polymer which is impermeable to (R)-dihydroetorphine, or a salt, or a hydrate thereof, and any other active ingredient present in the system (e.g. patch). Particularly preferred separating layers comprise a polyolefin (e.g. high and low density polyethylene, polypropylene), fluoropolymer (e.g. polytetrafluoroethylene), nylon, cellulose derivatives, ethylene-vinyl acetate, vinyl acetate, polyvinylchloride, polyurethane, polyesters (e.g. polyethylene phthalate, polyethylene terephthalate, polybutylene terephthalate or polyethylene naphthalate), and laminates of the afore-going.

The thickness of the drug-containing layer is preferably 20-150 microns, more preferably 30 to 120 microns and still more preferably 40-100 microns. A drug-containing layer thickness of less than 20 microns will tend to result in insufficient flux of drug through the skin and a thickness of greater than 150 microns will render the system (e.g. patch) too thick to be attractive to wear and use.

The backing layer can be any appropriate thickness which will provide the desired protective and support functions. Desirable materials and thicknesses will be apparent to the skilled man but may be in the range 40 to 70 microns. Similarly the removable release liner can be any appropriate thickness which will provide the necessary protection to the adhesive layer prior to application. Desirable materials and thicknesses will be apparent to the skilled man but may be in the range 80 to 120 microns. The skilled man will readily determine suitable thicknesses for any separating and/or adhesives layers present in the transdermal system (e.g. patch).

The total thickness of the system (e.g. patch) is preferably 100 to 350 microns, more preferably 150 to 300 microns and still more preferably 200 to 250 microns.

Preferred transdermal systems (e.g. patches) of the present invention have a skin contacting surface area of 2 to 64 cm², more preferably 4 to 64 cm² and still more preferably 6.25 to 36 cm². The system (e.g. patch) may be formed into any shape, e.g. as a square, rectangle, circle or oval. The system (e.g. patch) may also have a non-geometric shape.

In preferred transdermal systems (e.g. patches) of the present invention the concentration of (R)-dihydroetorphine, or salt or hydrate thereof, is 0.01 to 0.50 mg/cm², more preferably 0.1 to 0.45 mg/cm² and still more preferably 0.2 to 0.4 mg/cm². In further preferred transdermal systems (e.g. patches) the concentration of (R)-dihydroetorphine, or salt or hydrate thereof, is 0.5 to 12 mg/system (e.g. patch), more preferably 1 to 10 mg/system (e.g. patch) and still more preferably 2 to 8 mg/system (e.g. patch).

The transdermal systems (e.g. patches) of the present invention are preferably 3 to 7 day systems (e.g. patches). This means that the systems (e.g. patches) can deliver a therapeutically effective amount of (R)-dihydroetorphine, or a salt, or a hydrate thereof, for 3-7 days before the system (e.g. patch) needs to be removed and a new system (e.g. patch) put on. Preferably the system (e.g. patch) of the invention is a 7 day system (e.g. patch). Such systems (e.g. patches) are highly desirable since the patient only needs to renew their system (e.g. patch) once per week. Hence preferred systems (e.g. patches), e.g. when applied to the skin of a patient, provides a therapeutically effective amount of (R)-dihydroetorphine, or a salt or hydrate thereof, for at least 72 hours and more preferably 72-168 hours.

Preferred systems (e.g. patches) of the invention have a steady state in vitro flux rate of (R)-dihydroetorphine or salt or hydrate thereof of 0.3 to 0.9 μg/cm²/h, more preferably 0.5 to 0.9 μg/cm²/h and still more preferably 0.7 to 0.9 μg/cm²/h during a period 22 to 72 hours when tested in a Franz cell using dermatomised human skin (e.g. as determined in the examples). Particularly preferred systems (e.g. patches) of the invention comprise 6.25 mg (R)-dihydroetorphine, or a salt, or a hydrate thereof and have a steady state in vitro flux rate of (R)-dihydroetorphine or salt or hydrate thereof of 0.3 to 0.9 μg/cm²/h, more preferably 0.5 to 0.9 μg/cm²/h and still more preferably 0.7 to 0.9 μg/cm²/h during a period 22 to 72 hours when tested in a Franz cell using dermatomised human skin (e.g. as determined in the examples).

Preferred systems (e.g. patches) of the present invention are stable to storage. Preferably the systems (e.g. patches) of the invention are physically stable. Preferably the systems (e.g. patches) of the invention are chemically stable.

Lack of physical stability may manifest in the occurrence of crystallisation of (R)-dihydroetorphine or a salt or hydrate thereof in the drug-containing layer which can be observed microscopically. Such crystallisation is undesirable because it is highly unlikely that once formed the crystals will redissolve in the matrix. Moreover when (R)-dihydroetorphine, or a salt, or a hydrate thereof, is in the form of crystals it cannot be delivered through the skin.

Preferred systems (e.g. patches) of the present invention are stable as indicated by no crystallisation of (R)-dihydroetorphine or a salt or hydrate thereof in the drug-containing layer (e.g. as determined by microscopic observation, preferably as described in the examples) during storage at 25° C. and 60% relative humidity in a sealed system for at least 1 week, more preferably 2 weeks and still more preferably 4 weeks. Under these conditions, the most preferred systems (e.g. patches) may be stable for up to, e.g. 52 weeks.

Preferred systems (e.g. patches) of the present invention are stable as indicated by no crystallisation of (R)-dihydroetorphine or a salt or hydrate thereof in the drug-containing layer (e.g. as determined by microscopic observation, preferably as described in the examples) during storage at 40° C. and 75% relative humidity in a sealed system for at least 1 week, more preferably 2 weeks and still more preferably 4 weeks. Under these conditions, the most preferred systems (e.g. patches) may be stable for up to, e.g. 52 weeks.

Preferred systems (e.g. patches) of the present invention are stable as indicated by no crystallisation of (R)-dihydroetorphine or a salt or hydrate thereof in the drug-containing layer (e.g. as determined by microscopic observation, preferably as described in the examples) during storage at 40° C. and 75% relative humidity in an open system for at least 1 week, more preferably 2 weeks and still more preferably 4 weeks. Under these conditions, the most preferred systems (e.g. patches) may be stable for up to, e.g. 52 weeks.

Further preferred systems (e.g. patches) of the present invention are stable as indicated by no crystallisation of (R)-dihydroetorphine or a salt or hydrate thereof in the drug-containing layer (e.g. as determined by microscopic observation, preferably as described in the examples) during storage at 6-8° C. in a sealed system for at least 1 week, more preferably 2 weeks and still more preferably 4 weeks. Under these conditions, the most preferred systems (e.g. patches) may be stable for up to, e.g. 52 weeks.

Preferred systems (e.g. patches) of the present invention are stable as indicated by no crystallisation of (R)-dihydroetorphine or a salt or hydrate thereof in the drug-containing layer (e.g. as determined by microscopic observation, preferably as described in the examples) during storage at 60° C. in a sealed system for at least 6 days. Under these conditions, the most preferred systems (e.g. patches) may be stable for up to, e.g. 30 days.

Preferred systems (e.g. patches) of the present invention adhere to human skin for at least 72 hours, more preferably at least 120 hours and still more preferably at least 168 hours. The systems (e.g. patches) may, for example, adhere to human skin for 72 to 336 hours, more preferably 96 to 240 hours and still more preferably 120 to 168 hours.

The adhesion of a system (e.g. patch) may also be tested by measuring its peel strength from a stainless steel surface using a Zwick/Roell machine as described in the examples. The peel strength of systems (e.g. patches) of the invention comprising (R)-dihydroetorphine, or a salt, or a hydrate thereof, in their drug containing layer may be compared to identical systems (e.g. patches) but lacking (R)-dihydroetorphine or salt or hydrate thereof from the drug-containing layer. This enables the relative impact of the (R)-dihydroetorphine, or a salt, or a hydrate thereof, on the adhesiveness of the drug-containing layer to be determined. Preferred systems (e.g. patches) of the invention have a peel strength of ±30%, more preferably ±25% and still more preferably ±10% of an identical system except for the absence of (R)-dihydroetorphine or salt or hydrate thereof in its drug-containing layer.

In a further embodiment of the present invention the transdermal system (e.g. patch) comprises:
a drug-containing layer comprising (R)-dihydroetorphine, or a salt or a hydrate thereof, and a pressure sensitive adhesive; and
a backing layer;
wherein said system (e.g. patch) is a 3 to 7 day system (e.g. patch).

In a yet further embodiment of the present invention the transdermal system (e.g. patch) comprises:
a drug-containing layer comprising (R)-dihydroetorphine, or a salt or a hydrate thereof, and a pressure sensitive adhesive; and
a backing layer;
wherein said system (e.g. patch) (e.g. when applied to the skin of a patient) provides a therapeutically effective amount of (R)-dihydroetorphine, or a salt or hydrate thereof, for at least 72 hours.

In a yet further embodiment of the present invention the transdermal system (e.g. patch) comprises:
a drug-containing layer comprising (R)-dihydroetorphine, or a salt or a hydrate thereof, and a pressure sensitive adhesive; and
a backing layer;
wherein wherein no crystallisation of (R)-dihydroetorphine, or a salt or hydrate thereof, in the drug-containing layer (e.g. as determined by microscopic observation, preferably as described in the examples) occurs during storage at 60° C. in a sealed system for at least 1 week.

In these systems (e.g. patches) the pressure sensitive adhesive is preferably a polymer and more preferably a polymer selected from styrene-based block copolymers, polyvinyl acetates, poly(iso)butylenes, natural and synthetic rubbers, polyurethanes, polyisoprenes, organopolysiloxanes and poly(meth)acrylates. Still more preferably the pressure sensitive adhesive is selected from styrene-based block copolymers, polyisobutylenes, organopolysiloxanes and poly(meth)acrylates and yet more preferably organopolysiloxanes and poly(meth)acrylates. Poly(meth)acrylates are especially preferred.

Representative examples of suitable adhesives are those described above.

The systems (e.g. patches) of the present invention may be prepared using conventional methods. For instance the system (e.g. patch) may be prepared by coating a backing layer with a solution of (R)-dihydroetorphine or a salt or hydrate thereof and pressure sensitive adhesive, e.g. poly(meth)acrylate, in a solvent, removing the solvent from the coated layer to form the drug-containing layer and applying a release liner thereon. In an alternative method the system (e.g. patch) is prepared by coating a release liner with a solution of (R)-dihydroetorphine or a salt or hydrate thereof and pressure sensitive adhesive, e.g. poly(meth)acrylate, in a solvent, removing the solvent from the coated layer to form the drug-containing layer and applying a backing layer thereon. Preferred methods further comprise a step of cutting the resulting layered structure into the desired size and/or shape. Preferred solvents for the preparation of the solution of (R)-dihydroetorphine, or a salt, or hydrate thereof, include ethylacetate, hexane, heptane, acetylacetone, toluene, isopropanol, methanol and mixtures thereof. Ethylacetate is a particularly preferred solvent. The preferred drying conditions for removal of the solvent in the coated drug-containing layer are 60 to 120° C. for e.g. 5 to 30 minutes.

The systems (e.g. patches) of the present invention may be used in medicine and particularly for the treatment of pain. A method for the treatment of pain in patient in need thereof comprises: applying a system (e.g. patch) as hereinbefore described to the subject. The system (e.g. patch) transdermally delivers a therapeutic amount of (R)-dihydroetorphine, or a salt, or a hydrate thereof, through the skin to the bloodstream. Preferably the system (e.g. patch) is applied for at least 72 hours.

### DETAILED DESCRIPTION OF THE INVENTION

Referring to FIG. 1a, it shows a transdermal patch of the present invention that is ready to be placed on the skin of a patient. The patch 1 is a laminate of two layers. A top backing layer 2, which is substantially impermeable to (R)-dihydroetorphine, and a drug layer 3, which comprises (R)-dihydroetorphine or a salt or a hydrate thereof and a poly(meth)acrylate. The backing layer 2 defines the top of the patch and serves as a protective cover for the drug layer 3.

Referring to FIG. 1b, it shows a transdermal patch of the present invention in a form suitable for packaging and storage. The patch 10 is a laminate of three layers. A top backing layer 2, a drug layer 3 comprising (R)-dihydroetorphine or a salt or a hydrate thereof and a poly(meth)acryalte adhesive and a removable release liner 4. Prior to use, the removable release liner 4 is removed to expose the drug layer 3 comprising adhesive. This is applied to the skin of a patient.

FIGS. 2a, 2b and 2c each show alternative patch structures in a form suitable for packaging and storage.

FIG. 2a shows another transdermal patch comprising a single drug layer. Compared to the patch in FIG. 1b, however, the patch comprises an additional adhesive layer 6 and a separating layer 5. The separating layer 5 is formed on top of the drug-containing layer 3 and the adhesive layer 6 is formed around the resulting structure. Thus the adhesive layer 6, together with release liner 4, encompasses or encapsulates the drug-containing layer 3 and the separating layer 5. The backing layer 2 is formed on top of the adhesive layer 6. The release liner 4 contacts the underside of the drug-containing layer 3 and the adhesive layer 6 that surrounds the drug-containing layer. In this arrangement, the drug-containing layer may comprise a reservoir of, for example, a solution of the drug. In this case, there would typically be a membrane 7 through which, in use, the drug passes to reach the skin.

FIG. 2b shows a patch comprising multiple drug layers. Thus the patch comprises a top backing layer 2, a first drug layer comprising (R)-dihydroetorphine or a salt or a hydrate thereof and a poly(meth)acrylate adhesive 6, a separating layer (a rate limiting membrane) 5, a second drug containing layer comprising a drug and a pressure senstitive adhesive 3 and a release liner 4. The drug may optionally be (R)-dihydroetorphine or a salt or a hydrate thereof.

FIG. 2c shows a patch comprising a top backing layer 2, a drug-containing layer comprising (R)-dihydroetorphine or a salt or hydrate thereof and poly(meth)acrylate 3, a separating layer (a rate limiting membrane) 5, an adhesive layer 6 and a release liner 4.

### BRIEF DESCRIPTION OF THE DRAWINGS

FIGS. 1a and 1b show schematics of transdermal patches of the invention;

FIGS. 2a, 2b and 2c show schematics of alternative transdermal patches of the invention;

FIG. 3 is a plot of mean plasma concentration (pg/ml) of (R)-DHE versus time (hrs); and

FIG. 4 is a plot of log mean plasma concentration (pg/ml) of (R)-DHE versus time (hrs).

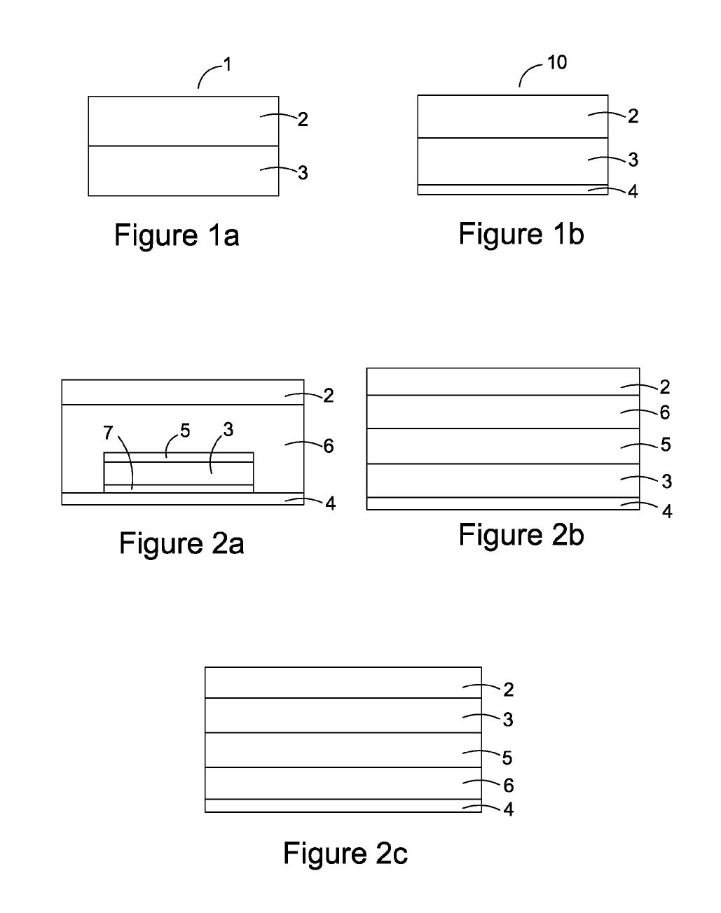

### EXAMPLES

Manufacture of transdermal patch comprising (R)-DHE

Patches of 25 cm² size with a load of 0.25 mg (R)-DHE/cm² (total drug load 6.25 mg/patch) were prepared.

(R)-DHE was weighted to a calculated 4.5% drug load (R)-DHE in the dried patch matrix of DURO-TAK 87-9301 (from Henkel) and dissolved in ethylacetate. The matrix solvent system was stirred for 30 minutes on a magnetic stirrer to yield a homogenous mixture. After mixing, the drug/polymer mixture was hand cast onto a release liner (Loparex Prime Liner FL 2000). Casting was carried out with a casting knife of variable width to achieve a target dry area weight matrix of 55 g/m². The cast was then dried at room temperature for 10 minutes, transferred to a convection oven and dried at 70° C. for 15 minutes and at 100° C. for 5 minutes. Finally the dried casts were hand-laminated with an occlusive backing, Scotchpak 9738, and patches were cut out of the laminate with a cutting die.
Assessment of Pharmacokinetic profile of transdermally delivered (R)-DHE

The aim of the study was:
To characterise the PK profile of (R)-dihydroetorphine as a 7-day transdermal delivery system formulation (R-DHE TDS).
To assess PK dose-proportionality of different doses of R-DHE TDS.

### Overall Study Design and Plan

The study was an open-label, dose-ascending, single-period, single-dose pilot study in which subjects received R-DHE TDS under naltrexone cover. Six cohorts of 6 subjects received R-DHE in a 7-day patch formulation (R-DHE TDS). Proposed dose levels of R-DHE TDS were determined from plasma level estimations from a reference study.

Review of PK and safety data took place on completion of each dose level to determine dose escalation for the next cohort of subjects. Planned dose levels of 1/4, 1/2, 1, 2, 4, and 6 patches of R-DHE TDS were applied for 7-day wear. The maximum dose level was 6 R-DHE patches.

Subjects were confined to the study unit from check-in on the day before Investigational Medicinal Product (IMP) administration (Day −1) until post-dose assessments (pharmacokinetic and safety measurements) were completed at 192 hours post-patch application (Day 9). Subjects returned to the study unit at 204 and 216 hours (Day 9 evening and Day 10 morning) for final post-dose assessments.

Safety was assessed by documentation of spontaneously reported adverse events, clinical laboratory results, vital signs, physical examinations, pulse oximetry (SpO₂), questionnaires, duration of patch wear observations and 12-lead ECGs.

In case of subject discontinuation (prior to patch removal on Day 8), where possible, subjects remained in the study unit for 24 hours post-patch removal and scheduled PK and safety measurements were taken during this time.

All subjects returned to the study unit for a post-study medical visit 7 days from patch removal.

Subjects who received naltrexone only but no IMP (i.e. reserve subjects, or subjects who were discontinued prior to IMP administration and who were replaced) had a post-study medical before discharge from the study unit.

### Indication and Criteria for Inclusion

Healthy males, 18-45 years inclusive, free of significant abnormal findings as determined by medical history, physical examination, vital signs, laboratory tests and ECG and whose primary care physician had confirmed within the last 12 months that there was nothing in their medical history that would preclude their enrolment into a clinical study.

### Pharmacokinetic Sample Collection

Blood Samples were Collected as Follows:

Pre-dose and at 0.5, 1, 1.5, 2, 3, 4, 5, 6, 8, 12, 16, 24, 36, 48, 60, 72, 84, 96, 108, 120, 144, 168 hours after patch application and 1, 4, 8, 12, 24, 36 and 48 hours after patch removal (30 samples per dosing period). Approximately 180 mL of blood (6 mL on 30 occasions) was taken from a subject for PK measurements. In case of subject discontinuation prior to patch removal on Day 8 subjects, where possible, remained in the study unit for 24 hours post-patch removal and blood samples were collected as close as possible to the post-patch removal scheduled time points during this time.

Venous blood samples (6 mL each) were drawn into tubes containing K₂EDTA anticoagulant. Samples were centrifuged within 30 minutes of collection. Following centrifugation (1500 G, 4° C., 15 minutes), the plasma was transferred via pipette into 2 labelled polypropylene tubes and stored at −20° C. within one hour of collection.

PK data were analysed and reviewed on an ongoing basis during the dose escalation periods to assess plasma concentration levels and provide additional information for dose escalation decisions.

### Pharmacokinetic Parameters

Plasma concentrations of (R)-dihydroetorphine were analysed to determine the following PK parameters:
AUCt, AUCt/D, AUCINF, AUCINF/D, Cmax, Cmax/D, tmax, LambdaZ, t1/2Z, Ctau, Ctau/D and flux (where D=adjusted for dose. For this study, dose was defined as the nominal patch content, i.e. 6.25 mg per whole patch). Flux was calculated using the residual patch content. Flux is described as the rate of transfer of drug from the patch to the systemic circulation, and is estimated as the difference between the nominal pre-application quantity and the residual (post-study) quantity of drug, divided by the actual duration of patch wear in hours. The plasma concentrations recorded immediately before patch removal (Ctau, Ctau/D) were also reported.

Areas under the plasma concentration-time curve were calculated from the time of dosing up to the final observed plasma concentration (AUCt) using the log-linear trapezoidal method. Where possible, the terminal phase rate constants (LambdaZ) were estimated using those points determined to be in the terminal log-linear phase. Half-lives (t1/2Z) were determined from the ratio of In 2 to LambdaZ. The areas under the plasma concentration-time curve between the last measured point and infinity were calculated from the ratio of the final observed plasma concentration (Clast) to LambdaZ. This was added to the AUCt to yield the area under the plasma concentration-time curve between the time of administration and infinity (AUCINF).

For the non-compartmental analysis of plasma concentration data, all pharmacokinetic calculations were performed using Phoenix WinNonlin, Version 6.2 or later, using actual sample times. Dose-adjusted parameters were calculated during statistical analysis. Pharmacokinetic parameters were calculated using actual elapsed times. Where actual elapsed times were not available, nominal times were substituted for that time point.

### Safety Assessments

The obligations and responsibilities with regards to collection, distribution and onward reporting of adverse events and reactions to the appropriate regulatory bodies, committees and other investigators (including SUSAR reporting) were carried out in accordance with local regulations. Safety was assessed by documentation of spontaneously reported adverse events, clinical laboratory results, vital signs, physical examinations, pulse oximetry (Sp02), questionnaires and 12-lead ECGs.

### Investigational Drug

R-DHE TDS patches 6.25 mg (manufactured by Labtec GmbH, Germany) were applied to the upper back. 1/4, 1/2, 1, 2, 4, and 6 patches of R-DHE TDS were applied for 7 days of continuous wear. Dose level may have been adjusted based on safety and PK data review after each cohort. The total number of patches worn by a subject would not exceed 6 patches.

Non-Investigational Medicinal Product(s) (NIMP(s))

Naltrexone hydrochloride tablet 50 mg (Nalorex® tablets, Bristol-Myers Squibb Pharmaceuticals Limited). Naltrexone tablets 50 mg were orally administered 12-hourly from the evening of Day −1 (13 hours before patch administration) until 11 hours after R-DHE TDS patch removal on Day 8 (17 occasions in total). Dosage may have been increased to 2×50 mg if deemed necessary by the Investigator. If a subject discontinued the study and patch removal was prior to Day 8, a naltrexone dose was administered 11 hours after R-DHE TDS patch removal, or at a time judged to be appropriate by the Investigator before the subject was discharged from the study unit.

### Method of Administration

### Patch Application:

Clipped excessive hair (did not shave), cleaned site with clean water (did not use alcohol, oils, lotions, soaps or abrasive devices), and allowed skin to completely dry.

Cut open foil pouch (used scissors carefully to avoid damaging the patch), tore pouch open, removed the patch from foil pouch. The foil pouch containing the patch was opened immediately prior to application. The opened pouch was not discarded.

Folded back half of the patch liner (backing) and grasped the other half, taking care not to touch the adhesive.

Applied the patch to the left and/or right upper back, removed the liner. The liner was not discarded. If the person applying the patch inadvertently touched the adhesive part behind the protective liner, they washed the affected area with water. Soaps, lotions, alcohol or other solvents were not used as these may have facilitated drug transfer through the skin.

Pressed down on the patch with the palm of your hand for 30 seconds, making sure contact was complete, especially round the edges (do not rub).

Placed the opened foil pouch and liner into a separate, clean plastic bag, sealed and labelled for storage until the time of patch removal. If multiple patches were applied for a single dosing, a single plastic bag was to be used for storage of all individual bags containing each used patch, liner and pouch.

Washed hands with clean water after patch application and handling of materials was complete.

If the planned dosing schedule required the patch to be cut, the patch was divided appropriately (e.g. in half) by measurement with a ruler and lightly marked with a pencil, and then cut with scissors. After cutting, the scissors were wiped with a sterile wipe and the wipe was discarded as clinical waste after use. The unused portion of the patch was placed into a separate, clean plastic bag, sealed and labelled for storage.

If the planned dosing schedule required multiple patches to be applied, the patches were applied so as not to overlap. Patches may have been applied to the left and/or right upper back.

Whilst the patch was applied, subjects may have had a shower but must have refrained from washing, or rubbing the site of patch application. Subjects refrained from showering until the day after patch application.

### Patch Removal:

R-DHE TDS was removed on the morning of Day 8 following the blood draw at 168 hours after patch application.

Subjects' skin at the site of patch application was wiped with a sterile wipe after patch removal to remove any residual traces of drug. The skin wipe was included with the used patch for residual analysis.

After removal, each patch was placed on the original release liner and into the original pouch and then placed in a separate, clean plastic bag, sealed and labelled. The patch was not folded. The used patch was maintained at room temperature at the study site until being shipped to the analytical laboratory for residual analysis.

### Patch Adherence:

If at any time the edges of the patch began to peel off, the edges were taped down with suitable skin tape (e.g. Tegaderm™). Any occurrence of a patch becoming loose was documented.

### Concomitant Therapies

All medications not prohibited by the protocol and considered necessary for the subject's welfare were administered and/or continued under the supervision of the Investigator. For subjects who received study treatments, concomitant therapies, including over-the-counter medications, that were ongoing as of the date of informed consent were recorded on the Concomitant Therapy section of the CRFs. The doses of these concomitant medications taken during the treatment period were kept constant until study completion. Any significant non-pharmacological therapies and/or procedures initiated during the study, beginning as of the date of informed consent were also recorded. The use of such concomitant medications was approved in advance by the Sponsor, when possible. The Investigator recorded the AE for which the concomitant medication was administered on the CRF.

Paracetamol was permitted for the treatment of headache or other symptoms as appropriate.

Naloxone injection was available for emergency use for respiratory depression.

Granisetron was permitted for the treatment of nausea and vomiting, although other treatments may have been used if considered more appropriate by the Investigator.

### Bioanalytical Data Management and Quality Control

Analysis of plasma samples was performed using a validated analytical method based on liquid chromatography-tandem mass spectrometry (LC-MS/MS). The samples were analysed over the calibration range of 5.00-5000 pg/mL. The method was originally validated at the former Quotient Bioresearch, now Quotient Bio Analytical Sciences.

Residual analysis of transdermal patches and gauze was performed using a validated liquid chromatography-ultra violet (LC-UV) analytical method. Analysis was carried out over the calibration range 3.125-75 μg/mL for R-DHE.

### Statistical Methods

All data analyses were performed by the Sponsor after the study was completed and the database was locked. Statistical programming and analyses were performed using SAS® version 9.1.3 (SAS Institute, Cary, N.C. 27513).

### Clinical Pharmacology Results

### Pharmacokinetics

The primary pharmacokinetic (PK) objectives of this study were to characterise the PK profile of R-DHE as a 7-day transdermal delivery system formulation (R-DHE TDS) and to assess the PK dose-proportionality of different doses of R-DHE TDS.

Analyses of PK parameters were performed using data from all the subjects in the PK population. One subject was deemed as having a major protocol deviation, with R-DHE TDS removal after 28.2 hours of application due to them being discontinued from the study. No additional exclusions were made from the PK population on the basis of R-DHE TDS adhesion, as all subjects had >50% R-DHE TDS adhesion on any day of R-DHE TDS wear.

### Plasma Concentration—Time Curves

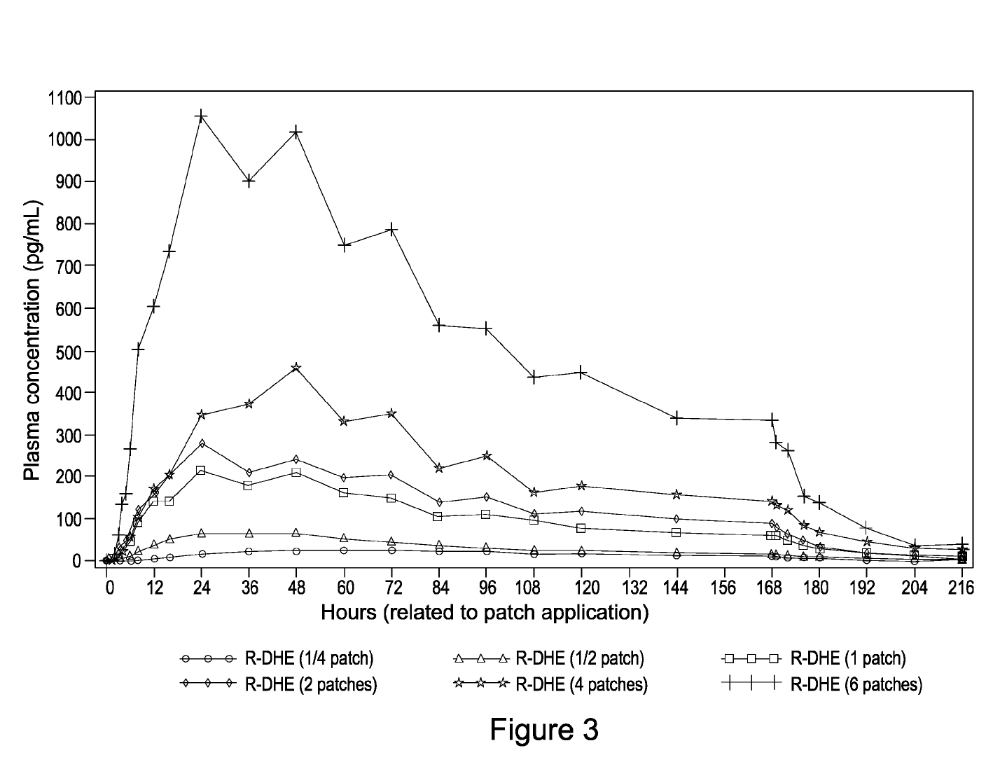

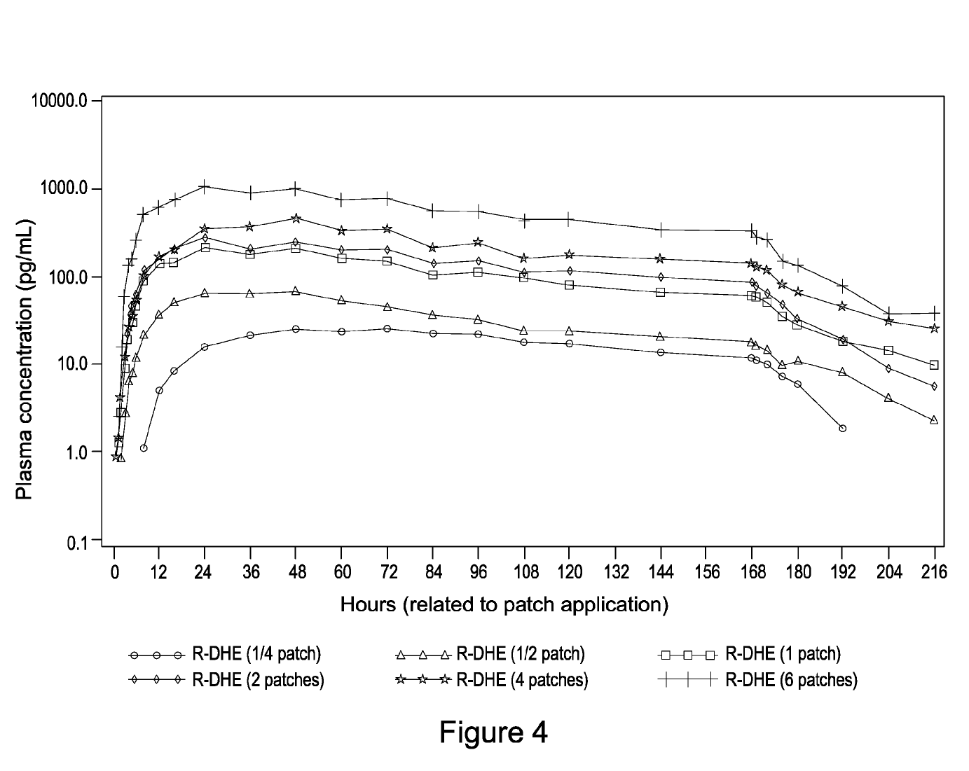

Mean observed plasma concentration-time curves for R-DHE are presented on linear scales in FIG. 3 and log-linear scales in FIG. 4. Table 1 below presents the PK summary statistics for R-DHE. Statistical results making an exploratory comparison of R-DHE doses (test versus reference) based on 1 patch (6.25 mg) as a reference, using dose-adjusted parameters are displayed in Table 2 below (secondary plasma PK parameters).

Plasma concentrations increased with increasing R-DHE TDS dose level. AUCt values ranged from 2932.74 pg.h/mL (geometric mean) for the 1/4 R-DHE TDS to 100394.41 pg.h/mL for administration of 6 R-DHE TDS. AUCINF values were similar, ranging from 2783.08 pg.h/mL to 102903.35 pg.h/mL. Mean Cₘₐₓ values ranged from 27.85 pg/mL to 1072 pg/mL across the dose levels. Mean Cₜₐᵤ also increased with increasing dose level, from 11.24 pg/mL to 323.6 pg/mL from the 1/4 to 6 R-DHE TDS (Table below).

Ratios for the statistical comparison of dose levels for AUCt/D were 72.36%, 75.03%, 78.53%, 64.25% and 103.22% for the 1/4, 1/2, 2, 4, and 6 R-DHE TDS, using the 1 R-DHE TDS as the reference for the comparisons. For Cmax/D, ratios were 61.23%, 77.41%, 74.21%, 61.88% and 98.22% for the 1/4, 1/2, 2, 4, and 6 R-DHE TDS.

Estimates of the slope parameter for the power model test for dose proportionality were approximately 1 (1.042 for AUCt, 1.064 AUCINF, 1.057 for Cmax and 1.034 for Ctau).

The median tmax was 72 hours for the 1/4 R-DHE TDS dose level, 24 hours for the 2 R-DHE TDS and 6 R-DHE TDS dose levels, and was 48 hours for the 1 and 4 R-DHE TDS dose levels and was 42 hours for the 1/2 R-DHE TDS. Half-life was shortest for the 1/4 R-DHE TDS dose level at 11.565 hours and longest for the 1/2 R-DHE TDS at 36.041 hours. Mean half-life was similar for the 1 and 2 R-DHE TDS dose levels at 14.074 (only one value available) and 13.751 hours respectively and was 17.857 for the 6 R-DHE TDS and 25.786 hours for the 4 R-DHE TDS dose level (Table below).

### Flux

Mean flux rates increased with increasing dose level and were 3.8 μg/h, 6.82 μg/h, 7.88 μg/h, 14.93 μg/h, 33.05 μg/h and 50.72 μg/h for the 1/4, 1/2, 1, 2, 4 and 6 R-DHE TDS dose levels respectively (Table 3 below).

TABLE 1

| | | R-DHE | | | |
|---|---|---|---|---|---|
| PK | | ¼ patch | ½ patch | 1 patch | 2 patches |
| Parameter | Statistics | (N = 6) | (N = 6) | (N = 5) | (N = 6) |
| AUCt | n (n*) | 6 (6) | 6 (6) | 5 (5) | 6 (6) |
| (pg.h/mL) | Geometric Mean | 2932.74 | 6081.35 | 16210.85 | 25459.36 |
| | Log SD/SE | 1.365, 1.136 | 1.519, 1.186 | 2.285, 1.447 | 1.459, 1.167 |
| | CV (%) | 31.9 | 43.7 | 99.0 | 39.2 |
| | Arithmetic | 3049.66 | 6546.13 | 20871.99 | 26808.75 |
| | Mean SD/SE | 904.524/369.270 | 2803.159/1144.385 | 15505.942/6934.468 | 8253.271/3369.384 |
| | Median | 3034.06 | 6040.35 | 21800.33 | 28663.04 |
| | Min, Max | 1894.0, 4088.9 | 3745.0, 11188.7 | 6817.9, 44512.4 | 12651.4, 34404.8 |
| Cmax | n (n*) | 6 (6) | 6 (6) | 5 (5) | 6 (6) |
| (pg/mL) | Geometric Mean | 27.85 | 70.42 | 181.9 | 270.0 |
| | Log SD/SE | 1.3276, 1.1227 | 1.5430, 1.1937 | 2.2600, 1.4400 | 1.4594, 1.1669 |
| | CV (%) | 28.92 | 45.49 | 97.17 | 39.19 |
| | Arithmetic Mean | 28.78 | 75.95 | 231.8 | 286.2 |
| | SD/SE | 8.0029/3.2672 | 31.394/12.817 | 163.96/73.326 | 104.93/42.839 |
| | Median | 29.50 | 70.50 | 223.0 | 261.0 |
| | Min, Max | 20.0, 41.0 | 37.8, 121 | 75.6, 461 | 153, 438 |
| Ctau | n (n*) | 6 (6) | 6 (6) | 5 (5) | 6 (6) |
| (pg/mL) | Geometric Mean | 11.24 | 16.59 | 49.76 | 84.98 |
| | Log SD/SE | 1.3737, 1.1384 | 1.5322, 1.1903 | 2.0729, 1.3854 | 1.3643, 1.1352 |
| | CV (%) | 32.57 | 44.69 | 83.74 | 31.83 |
| | Arithmetic | 11.74 | 17.93 | 60.74 | 88.08 |
| | Mean SD/SE | 3.8829/1.5852 | 7.9241/3.2350 | 40.486/18.106 | 23.153/9.4522 |
| | Median | 10.30 | 14.80 | 63.00 | 93.70 |
| | Min, Max | 8.29, 17.0 | 10.2, 28.8 | 22.2, 121 | 47.8, 110 |
| tmax | n | 6 | 6 | 5 | 6 |
| (h) | Arithmetic Mean | 66.0028 | 38.0000 | 40.8000 | 30.0000 |
| | SD/SE | 21.13291/8.62748 | 11.79830/4.81664 | 16.09969/7.20000 | 10.03992/4.09878 |
| | Median | 72.0000 | 42.0000 | 48.0000 | 24.0000 |
| | Min, Max | 36.000, 96.017 | 24.000, 48.000 | 24.000, 60.000 | 24.000, 48.000 |
| LambdaZ | n | 2 | 4 | 1 | 5 |
| (h⁻¹) | Arithmetic Mean | 0.0600 | 0.0225 | 0.0492 | 0.0510 |
| | SD/SE | 0.00353/0.00250 | 0.00914/0.00457 | | 0.00589/0.00264 |
| | Median | 0.0600 | 0.0237 | 0.0492 | 0.0520 |
| | Min, Max | 0.058, 0.063 | 0.011, 0.031 | 0.049, 0.049 | 0.041, 0.057 |
| t1/2Z | n | 2 | 4 | 1 | 5 |
| (h) | Arithmetic Mean | 11.565 | 36.041 | 14.074 | 13.751 |
| | SD/SE | 0.6800/0.4808 | 17.7913/8.8957 | | 1.7875/0.7994 |
| | Median | 11.565 | 30.560 | 14.074 | 13.321 |
| | Min, Max | 11.08, 12.05 | 22.14, 60.91 | 14.07, 14.07 | 12.12, 16.82 |

| | | R-DHE | |
|---|---|---|---|
| PK | | 4 patches | 6 patches |
| Parameter | Statistics | (N = 6) | (N = 6) |
| AUCt | n (n*) | 6 (6) | 6 (6) |
| (pg.h/mL) | Geometric Mean | 41659.29 | 100394.41 |
| | Log SD/SE | 1.285, 1.108 | 1.351, 1.131 |
| | CV (%) | 25.5 | 30.7 |
| | Arithmetic | 42670.40 | 103953.49 |
| | Mean SD/SE | 9434.372/3851.566 | 28038.277/11446.578 |
| | Median | 42709.13 | 105471.12 |
| | Min, Max | 26139.5, 53409.4 | 59413.6, 139805.5 |
| Cmax | n (n*) | 6 (6) | 6 (6) |
| (pg/mL) | Geometric Mean | 450.3 | 1072 |
| | Log SD/SE | 1.4121, 1.1513 | 1.3708, 1.1374 |
| | CV (%) | 35.56 | 32.34 |
| | Arithmetic Mean | 471.5 | 1116 |
| | SD/SE | 147.14/60.071 | 336.40/137.33 |
| | Median | 476.5 | 1110 |
| | Min, Max | 247, 692 | 689, 1530 |
| Ctau | n (n*) | 6 (6) | 6 (6) |
| (pg/mL) | Geometric Mean | 139.6 | 323.6 |
| | Log SD/SE | 1.0890, 1.0354 | 1.2955, 1.1115 |
| | CV (%) | 8.54 | 26.33 |
| | Arithmetic | 140.0 | 332.5 |
| | Mean SD/SE | 12.116/4.9464 | 82.133/33.531 |
| | Median | 138.0 | 334.0 |
| | Min, Max | 127, 159 | 220, 432 |
| tmax | n | 6 | 6 |
| (h) | Arithmetic Mean | 46.0000 | 32.0000 |
| | SD/SE | 9.03327/3.68782 | 12.39355/5.05964 |
| | Median | 48.0000 | 24.0000 |
| | Min, Max | 36.000, 60.000 | 24.000, 48.000 |
| LambdaZ | n | 3 | 3 |
| (h⁻¹) | Arithmetic Mean | 0.0315 | 0.0418 |
| | SD/SE | 0.01305/0.00753 | 0.01485/0.00857 |
| | Median | 0.0362 | 0.0348 |
| | Min, Max | 0.017, 0.041 | 0.032, 0.059 |
| t1/2Z | n | 3 | 3 |
| (h) | Arithmetic Mean | 25.786 | 17.857 |
| | SD/SE | 13.6622/7.8879 | 5.3494/3.0885 |
| | Median | 19.146 | 19.917 |
| | Min, Max | 16.71, 41.50 | 11.78, 21.87 |

N: Number of subjects in population.
n: Number of subjects with data available.
n*: Number of subjects with non-zero data available. This is used to calculate geometric mean, log SD/SE, and CV.
CV: Coefficient of Variation. Calculated on the log-transformed data as sqrt(exp(sigma\hat{ }2 − 1) × 100
Lambda and t1/2Z values are excluded if R2 <0.85 or non-consecutive points for Lambda Z estimate.

TABLE 2
Statistical comparison of Plasma PK Parameters: R-DHE TDS dose levels

| | Treatment Group | | | | Ratio (%) | |
|---|---|---|---|---|---|---|
| Parameter | (N) | n | LS Mean | LS Mean 90% CI | (Test/Reference) | 90% Confidence Interval |
| AUCt/D | ¼ R-DHE TDS patch 6.25 mg (6) | 6 | 1876.95 | (1385.01, 2543.64) | 72.36 | (46.10, 113.58) |
| | ½ R-DHE TDS patch 6.25 mg (6) | 6 | 1946.03 | (1435.98, 2637.25) | 75.03 | (47.80, 117.76) |
| | 1 R-DHE TDS patch 6.25 mg (5)* | 5 | 2593.74 | (1859.20, 3618.48) | | |
| | 2 R-DHE TDS patches 6.25 mg (6) | 6 | 2036.75 | (1502.92, 2760.19) | 78.53 | (50.03, 123.25) |
| | 4 R-DHE TDS patches 6.25 mg (6) | 6 | 1666.37 | (1229.62, 2258.26) | 64.25 | (40.93, 100.84) |
| | 6 R-DHE TDS patches 6.25 mg (6) | 6 | 2677.18 | (1975.50, 3628.11) | 103.22 | (65.76, 162.01) |
| Cmax/D | ¼ R-DHE TDS patch 6.25 mg (6) | 6 | 17.82 | (13.07, 24.31) | 61.23 | (38.64, 97.04) |
| | ½ R-DHE TDS patch 6.25 mg (6) | 6 | 22.53 | (16.52, 30.74) | 77.41 | (48.85, 122.68) |
| | 1 R-DHE TDS patch 6.25 mg (5)* | 5 | 29.11 | (20.72, 40.90) | | |
| | 2 R-DHE TDS patches 6.25 mg (6) | 6 | 21.60 | (15.84, 29.47) | 74.21 | (46.83, 117.62) |
| | 4 R-DHE TDS patches 6.25 mg (6) | 6 | 18.01 | (13.21, 24.57) | 61.88 | (39.05, 98.07) |
| | 6 R-DHE TDS patches 6.25 mg (6) | 6 | 28.59 | (20.96, 39.00) | 98.22 | (61.98, 155.66) |
| Ctau/D | ¼ R-DHE TDS patch 6.25 mg (6) | 6 | 7.19 | (5.49, 9.42) | 90.34 | (60.52, 134.83) |
| | ½ R-DHE TDS patch 6.25 mg (6) | 6 | 5.31 | (4.05, 6.95) | 66.68 | (44.67, 99.52) |
| | 1 R-DHE TDS patch 6.25 mg (5)* | 5 | 7.96 | (5.92, 10.70) | | |
| | 2 R-DHE TDS patches 6.25 mg (6) | 6 | 6.80 | (5.19, 8.91) | 85.39 | (57.21, 127.44) |
| | 4 R-DHE TDS patches 6.25 mg (6) | 6 | 5.58 | (4.26, 7.31) | 70.12 | (46.98, 104.66) |
| | 6 R-DHE TDS patches 6.25 mg (6) | 6 | 8.63 | (6.59, 11.31) | 108.39 | (72.62, 161.78) |

N: Number of subjects in population.
n: Number of subjects with data available
PK parameters were analysed using ANOVA with fixed terms for treatment. The ratio was calculated by transforming the difference between the natural log LS Means back to the linear scale.
*Reference treatment for this comparison.

TABLE 3

| | | R-DHE | | | | | |
|---|---|---|---|---|---|---|---|
| PK | | 1/4 patch | 1/2 patch | 1 patch | 2 patches | 4 patches | 6 patches |
| Parameter | Statistics | (N = 6) | (N = 6) | (N = 5) | (N = 6) | (N = 6) | (N = 6) |
| Flux | n | 6 | 6 | 5 | 6 | 6 | 6 |
| (μg/h) | Arithmetic Mean | 3.80 | 6.82 | 7.88 | 14.93 | 33.05 | 50.72 |
| | SD/SE | 1.769/0.722 | 6.397/2.612 | 5.153/2.305 | 10.926/4.460 | 17.492/7.141 | 20.636/8.425 |
| | Median | 4.25 | 4.50 | 8.20 | 14.60 | 31.05 | 52.10 |
| | Min, Max | 1.0, 5.7 | 0.7, 15.3 | 2.4, 14.5 | 3.2, 33.8 | 12.8, 55.8 | 28.1, 84.6 |

N: Number of subjects in population.
n: Number of subjects with data available.
n*: Number of subjects with non-zero data available. This is used to calculate geometric mean, log SD/SE, and CV.
CV: Coefficient of Variation. Calculated on the log-transformed data as sqrt(exp(sigma\hat{ }2 − 1) × 100
LambdaZ, t1/2Z, and AUCINF values are excluded if R2 <0.85 or non-consecutive points for Lambda Z estimate.

### Clinical Pharmacology Discussion and Conclusions

The primary PK objectives of this study were to characterise the PK profile of R-DHE as a 7-day transdermal delivery system formulation (R-DHE TDS) and to assess the PK dose-proportionality of different doses of R-DHE TDS.

Both Cₘₐₓ and AUC increased with ascending R-DHE TDS dose for R-DHE in this study. Dose-adjusted AUCt ranged from 1666.37 to 2677.18 pg.h/mL and dose-adjusted Cₘₐₓ from 17.82 pg/mL to 28.59 pg/mL were, broadly speaking, similar between dose levels, taking in to account the sample size. For most treatments (excluding the 1 R-DHE TDS dose level) CV % was between approximately 25-45%.

Flux rates were broadly proportional to the R-DHE TDS dose level (number or fraction of R-DHE TDS applied), especially between the 1, 2, 4 and 6 R-DHE TDS dose levels, which is encouraging from the prototype R-DHE TDS formulations used here for the first time in a Phase 1 clinical setting.

### Conclusions

Plasma concentrations and PK parameters for R-DHE increased reasonably proportionally with increasing doses of R-DHE TDS.

Dose adjusted AUCt and Cₘₐₓ were reasonably similar between the different dose levels.

Flux rates increased proportionally with increasing doses of R-DHE TDS, particularly between the 1, 2, 4 and 6 R-DHE TDS dose levels.

No deaths or SAEs occurred during the study and virtually all AEs experienced by subjects were mild and likely associated with opioid and/or naltrexone administration e.g. nausea.

Although one subject had a markedly abnormal haematology value and three subjects experienced clinically notable vital sign abnormalities, there were no notable changes in the number of subjects with abnormal values from pre-dose to post-dose, and no notable differences between the different dose cohorts.

Although there were two clinically significant ECG findings during the study, a separate cardiac report concluded that R-DHE TDS had no clinically relevant effects on cardiac repolarization or other ECG parameters.

In general, NAS, ARCI-49 and SOWS questionnaire results were unremarkable and did not reveal any particular safety concerns.

Results of this study show R-DHE TDS to be safe and well tolerated.

1. A transdermal delivery system comprising (R)-dihydroetorphine, or a salt, hydrate or derivative thereof, wherein said system has a rapid onset of (R)-dihydroetorphine plasma concentration characterised by the mean in vivo plasma concentration of (R)-dihydroetorphine achieving at least 50% of its Cₘₐₓ in less than 20 hours after application of the system to the skin of a human subject.

2. (canceled)

3. A system as claimed in claim 1, characterised by the mean in vivo plasma concentration of (R)-dihydroetorphine achieving at least 25% of its Cₘₐₓ in less than 10 hours after application of the system.

4. A system as claimed in claim 1, characterised by the mean in vivo plasma concentration of (R)-dihydroetorphine achieving at least 75% of its Cₘₐₓ in less than 24 hours after application of the system.

5. A system as claimed in claim 1, characterised by the mean in vivo plasma concentration of (R)-dihydroetorphine achieving Cₘₐₓ in less than 36 hours application of the system.

6. A system as claimed in claim 1, characterised by the mean in vivo plasma concentration of (R)-dihydroetorphine being at least 10 pg/mL in less than 12 hours after application of the system.

7. A system as claimed in claim 1, characterised by the mean in vivo plasma concentration of (R)-dihydroetorphine being at least 50 pg/mL in less than 14 hours after application of the system.

8. A system as claimed in claim 6, wherein said system is a single patch having a size of 25 cm² and comprising 6.25 mg of (R)-dihydroetorphine.

9. A system as claimed in claim 1, wherein the mean in vivo plasma concentration of (R)-dihydroetorphine increases at an average rate of 5 to 20 pg/ml/h until the mean in vivo concentration of (R)-dihydroetorphine reaches 50% of Cₘₐₓ, and when a single patch having a size of 25 cm² and comprising 6.25 mg of (R)-dihydroetorphine is applied.

10. A system as claimed in claim 1, wherein the mean in vivo plasma concentration of (R)-dihydroetorphine is 80 to 125% of 50 pg/ml in less than 8 hours after application of the system.

11. A system as claimed in claim 1, wherein the mean in vivo plasma concentration of (R)-dihydroetorphine is 80 to 125% of 100 pg/ml in less than 12 hours after application of the system.

12. A system as claimed in claim 1, wherein the mean in vivo plasma concentration of (R)-dihydroetorphine is 80 to 125% of 10 pg/ml in less than 6 hours after application of the system.

13. A system as claimed in claim 1, wherein said system has a rapid offset of (R)-dihydroetorphine plasma concentration characterised by the mean in vivo plasma concentration of (R)-dihydroetorphine reducing from its concentration at the time of removal of the system by at least 50% in less than 16 hours.

14. A system as claimed in claim 1, wherein said system has a rapid offset of (R)-dihydroetorphine plasma concentration characterised by the mean in vivo plasma concentration of (R)-dihydroetorphine reducing from its concentration at the time of removal of the system by at least 25% in less than 8 hours.

15. A system as claimed in claim 1, wherein the mean in vivo plasma concentration of (R)-dihydroetorphine is less than 50 pg/ml in less than 12 hours after removal of the system.

16. A system as claimed in claim 1, wherein the mean in vivo plasma concentration of (R)-dihydroetorphine is less than 10 pg/ml in less than 48 hours after removal of the system.

17. A system as claimed in claim 15, wherein said system is a single patch having a size of 25 cm² and comprising 6.25 mg of (R)-dihydroetorphine.

18. A system as claimed in claim 1, wherein the mean in vivo plasma concentration of (R)-dihydroetorphine is 80 to 125% of 80 pg/ml in less than 10 hours after removal of the system.

19. A system as claimed in claim 1, wherein the mean in vivo plasma concentration of (R)-dihydroetorphine is 80 to 125% of 50 pg/ml in less than 12 hours after removal of the system.

20. A system as claimed in claim 1, wherein the mean in vivo plasma concentration of (R)-dihydroetorphine is 80 to 125% of 40 pg/ml in less than 12 hours after removal of the system.

21. A system as claimed in claim 1, wherein the mean in vivo plasma concentration of (R)-dihydroetorphine is at least 50% of Cₘₐₓ for at least 72 hours after Cₘₐₓ is achieved.

22. A system as claimed in claim 1, wherein the mean in vivo plasma concentration of (R)-dihydroetorphine is at least 40% of Cₘₐₓ for at least 96 hours after application of the system.

23. A system as claimed in claim 1, wherein the mean in vivo plasma concentration of (R)-dihydroetorphine is at least 25% of Cₘₐₓ for at least 144 hours after application of the system.

24. A system as claimed in claim 1, wherein the mean in vivo plasma concentration of (R)-dihydroetorphine is at least 50 pg/ml for at least 72 hours, after Cₘₐₓ is achieved and when a single patch having a size of 25 cm² and comprising 6.25 mg of (R)-dihydroetorphine is applied.

25. A system as claimed in claim 1, which achieves a dose adjusted Cₘₐₓ of 80 to 125% of 180 pg/ml, relative to a single patch having a size of 25 cm² and comprising 6.25 mg of (R)-dihydroetorphine.

26. A system as claimed in claim 1, which achieves a dose adjusted AUCt of 80 to 125% of 16210 pg.h/ml, relative to a single patch having a size of 25 cm² and comprising 6.25 mg of (R)-dihydroetorphine.

27. A system as claimed in claim 1 having a mean in vivo flux rate of (R)-dihydroetorphine of 5 to 15 pg/h during a period of 168 hours, when a single patch having a size of 25 cm² and comprising 6.25 mg of (R)-dihydroetorphine is applied.

28. A system as claimed in claim 1, having a mean tₘₐₓ of 30 to 70 hours.

29. A system as claimed in claim 1 comprising:
a drug-containing layer comprising (R)-dihydroetorphine, or a salt or a hydrate thereof, and
a poly(meth)acrylate; and
a backing layer,

30. A system as claimed in claim 1 which is a transdermal patch.

31. A system as claimed in claim 1, wherein said (R)-dihydroetorphine is in free base form.

32. A system as claimed in claim 1, wherein said poly(meth)acrylate comprises at least two alkyl (meth)acrylate monomers.

33. A system as claimed in claim 31, wherein said alkyl (meth)acrylate monomers comprise 1 to 12 carbon atoms in the alkyl group.

34. A system as claimed in claim 32, wherein said poly(meth)acrylate consists of alkyl acrylate monomers and/or alkyl methacrylate monomers.

35. A system as claimed in any claim 1, wherein said drug-containing layer does not comprise a skin permeation enhancer.

36. (canceled)

37. (canceled)

38. A method for the treatment of pain in a human subject in need thereof comprising applying a system as claimed in claim 1 to the skin of said human subject.
